(19) United States
(12) Patent Application Publication
(10) Pub. No.: US 2006/0019366 A1
(43) Pub. Date: Jan. 26, 2006

(54) Reversibly modified thermostable enzyme compositions and methods of making and using the same

(76) Inventor: Wanli Bi, San Ramon, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/148,946

(22) Filed: Jun. 8, 2005

Related U.S. Application Data
(60) Provisional application No. 60/578,442, filed on Jun. 9, 2004.

(51) Int. Cl. C12Q 1/68; C12N 9/22

(52) U.S. Cl. 435/199; 435/6

(57) ABSTRACT

The present invention provides reversibly modified thermostable enzyme compositions and methods for making the same. The present invention also provides methods of using the reversibly modified thermostable enzyme compositions, as well as kits and systems comprising the reversibly modified thermostable enzymes.

### CROSS-REFERENCE

This application claims the benefit of U.S. Provisional Application No. 60/578,442, filed Jun. 9, 2004, which application is incorporated herein by reference.

### BACKGROUND OF THE INVENTION

Nucleic acid testing technologies, such as target amplification and signal amplification are widely used in clinical microbiology, blood screening, food safety, genetic disease diagnosis and prognosis, environmental microbiology, drug target discovery and validation, forensics, and other biomedical research. As such, nucleic acid testing is increasingly becoming an essential element of emerging pharmacogenomics, prenatal diagnoses, and molecular based cancer diagnoses and therapy. Therefore, the robustness of nucleic acid testing, specificity, sensitivity, reliability in terms of accuracy and precision, and affordability are of particular importance.

Nucleic acid sequence specific amplification allows sensitive detection of the presence or absence of a specific target nucleic acid sequence. Exemplary methods of thermocycling based target amplification include polymerase chain reaction (PCR) and ligase chain reaction (LCR). In contrast to thermocycling methods, isothermal amplification methods, which are carried out at a substantially constant temperature, can also be used for nucleic acid sequence specific amplification. Exemplary isothermal amplification methods include transcription-mediated amplification (TMA), nucleic acid sequence based amplification (NASBA), strand-displacement amplifcation (SDA), rolling circle amplification (RCA), single primer isothermal amplification (SPIA™), and exponential single primer isothermal amplification (X-SPIA™), self-sustained sequence replication (3SR) and loop mediated isothermal amplification (LAMP).

Since all enzymes, regardless their thermostability, are active in a range of temperatures, such property could adversely affect nucleic acid detection in terms of specificity, sensitivity and signal/noise ratio etc. This has been clearly demonstrated in PCR process. A thermostable DNA polymerase is essential for a PCR. Although the optimal temperature of catalytic activity of a thermostable DNA polymerase is around 60˜75° C., the DNA polymerase is also active at lower temperature. Therefore, the DNA polymerase retains a significant level of activity even at room temperature. Accordingly, the activity of the DNA polymerase at the lower temperature is a cause of non-specific amplification and reduced detection sensitivity.

“Hot-start” refers to an approach that inactivates thermostable enzymes, such as DNA polymerases, at low temperatures and restores the activity of the enzymes at an elevated temperature. Various hot-start methods have been developed to improve nucleic acid detection methods, including methods of using a physical barrier to separate the enzyme from the other components of the reaction, and chemical modification methods of inactivating the enzyme at lower temperatures.

A first method of reversible chemical modification to provide for inhibition of DNA polymerase activity at low temperature using a dicarboxylic acid anhydride is described in U.S. Pat. Nos. 5,677,152 and 5,773,258. In addition, a second method of reversible modification of a DNA polymerase using an aldehyde compound is described in U.S. Pat. No. 6,183,998 discloses. Both anhydride and aldehyde-mediated modifications form covalent bonds between the modifier compound and the DNA polymerase. Enzymatic activity is recovered by incubation of the modified enzyme in a proper buffer at high temperature.

However, the condition to reverse the modification of such methods is usually very harsh to the DNA polymerase. For example, with respect to aldehyde modified DNA polymerases, the aldehyde forms a Schiff base with amine group in the DNA polymerase. In order to achieve reactivation appropriately for PCR, the enzyme must be incubated at 95° C. for 15 minutes. Such a prolonged incubation period at 95° C. is very harmful to the enzyme activity and results in significant loss of activity.

In addition, activation of anhydride modified DNA polymerase is also harsh on the enzyme. In particular, the recommended activation condition for anhydride modified DNA polymerase is incubation at 95° C. for 10 minutes. In addition to the harsh conditions required for activation of the enzyme, the process for modification of the enzyme with the anhydride molecule is difficult because the anhydride molecule is generally very unstable in aqueous conditions and undergoes rapid hydrolysis, which destroys its ability to react with amine groups and thereby modify the target enzyme. Attempts of addressing the issue of hydrolysis have been proposed, such as performing the modification in an organic solvent as disclosed in U.S. Pat. No. 6,479,264. However, this modified process is long, tedious, and inefficient. More importantly, not all proteins are amenable to the treatment conditions.

Accordingly, there remains a need for development of a better chemical modification method that provides for improved reversibly modified enzymes that have improved sensitivity and specificity.

Relevant Literature

U.S. patents of interest include: U.S. Pat. Nos. 5,338,671, 5,411,876, 5,413,924, 5,427,930, 5,565,339, 5,643,764, 5,677,152, 5,773,258, 6,183,967, 6,183,998, 6,274,981, 6,403,341, 6,479,264, 6,511,810, 6,528,254, 6,548,250, 6,667,165, 6,191,278, 6,465,644, and 6,699,981. Literature of interest includes: Bae et al., 1999, Mol. Cells 9(1): 45-48; Barnes W. M., 1992, Gene 112: 29-35; Coleman et al., 1990, J. Chromatogr. 512: 345-363; Hoare et al., 1967, J. Biol. Chem. 242: 2447-2453; Hall et al., 2000, Proc. Natl. Acad. Sci. USA. 97(15): 8272-8277; Harrington et al., 1994, EMBO J. 13(5): 1235-1246; Henricksen et al., 2000, J. Biol. Chem. 275(22): 16420-16427; Hosfield et al., 1998, J. Biol. Chem. 273(42): 27154-17161; Kaiser et al., 1999, J. Biol. Chem. 274(30): 21387-21394; Lawyer et al., 1989, J Biol Chem. 1989 264(11): 6427-37; Lawyer et al., 1993, PCR Methods Appl. 2(4): 275-87; Leone et al., 1998, Nucleic Acids Res. 26(9): 2150-2155; Matsui et al., 1999, J. Biol. Chem. 274(26): 18297-18309; Murante et al., 1995, J. Biol. Chem. 270(51): 30377-30383; Nadeau et al., 1999, Anal. Biochem. 276: 177-187; Nieto et al., 1983, Biochim Biophys Acta. 749:204-10; Nilsson et al., 2002, Nucleic Acids Res. 30(14): e66; Palacian et al., 1990, Mol. Cell. Biochem. 97: 101-111; Rao et al., 1998, J. Bacteriol. 180(20): 5406-5412; Rumbaugh et al., 1999, J. Biol. Chem. 274(21): 14602-14608; Spears et al., 1997, Anal. Biochem. 247: 130-137; Staros et al., 1986, Anal. Biochem. 156: 220-222; Walker et al., 1996, Nucleic Acids Res. 24(2): 348-353; and Wu et al., 1996, Nucleic Acids Res. 24(11): 2036-2043.

### SUMMARY OF THE INVENTION

The present invention provides compositions of reversibly modified thermostable enzymes (e.g., thermostable DNA polymerase, thermostable RNA polymerase, thermostable nucleases, such as a thermostable endonuclease, thermostable ligases, thermostable RNase H, thermostable reverse transcriptase, thermostable helicases, thermostable RecA, etc.). Also provided are methods of producing the subject compositions using a carboxylic acid modifier reagent. The present invention also provides methods of using the reversibly modified thermostable enzyme compositions, as well as kits and systems comprising the reversibly modified thermostable enzyme compositions.

The invention provides for a thermostable enzyme composition, wherein the thermostable enzyme composition comprises a thermostable enzyme that has been covalently modified which results in essentially complete inactivation of enzyme activity, wherein incubation of said modified thermostable enzyme composition in an aqueous buffer, formulated to about pH 7 to about pH 9 at 25° C., at a temperature greater that about 50° C. results in at least a two-fold increase in activity of the composition in less than about 20 minutes. In some embodiments, the incubation of said thermostable enzyme composition in an aqueous buffer, formulated to about pH 7 to about pH 8 at 25° C., at a temperature greater than about 50° C. results in at least a two-fold increase in enzyme activity in less than about 20 minutes.

In some embodiments, the thermostable enzyme is a thermostable polymerase, such as a thermostable DNA polymerase or a thermostable RNA polymerase, a thermostable RNase H, a thermostable DNA nuclease, such as a thermostable DNA endonuclease, a thermostable DNA ligase, thermostable reverse transcriptase, thermostable helicase, thermostable RecA, and the like. In certain embodiments, the thermostable enzyme is a thermostable polymerase. In further embodiments, the thermostable polymerase is a thermostable DNA polymerase. In other embodiments, the thermostable polymerase is a thermostable RNA polymerase. In still other embodiments, the thermostable enzyme is a thermostable DNA nuclease, such as a thermostable DNA endonuclease. In other embodiments, the thermostable enzyme is derived from Thermus acquaticus, Thermus thermophilus, Thermatoga maritime, Aeropyrum pernix, Aquifex aeolicus, Archaeglobus fulgidus, Bacillus caldotenax, Carboxydothermus hydrogenformans, Methanobacterium thermoautotrophicum ΔH, Methanococcus jannaschii, Methanothermus fervidus, Pyrobaculum islandicum, Pyrococcus endeavori, Pyrococcus furiosus, Pyrococcus horihoshii, Pyrococcus profundus, Pyrococcus woesei, Pyrodictium occultum, Sulfolobus acidocaldarius, Sulfolobus solfataricus, Thermoanaerobacter thermohydrosulfuricus, Thermococcus celer, Thermococcus fumicolans, Thermococcus gorgonarius, Thermococcus kodakaraensis KOD1, Thermococcus litoralis, Thermococcus peptonophilus, Thermococcus sp. 9N-7, Thermococcus sp. TY, Thermococcus stetteri, Thermococcus zilligii, Thermoplasma acidophilum, Thermus brokianus, Thermus caldophilus GK24, Thermus flavus, Thermus rubens, or a mutant thereof.

In certain embodiments, the thermostable enzyme has been modified by a carboxylic acid modifier reagent described by the formula: 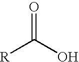
wherein R is a hydrogen, a substituted or unsubstituted phenyl group, a substituted or unsubstituted cycloalkyl group, a substituted or unsubstituted heteroaromatic group, or a substituted or unsubstituted alkyl group. In still further embodiments, the carboxylic acid modifier reagent is citraconic acid or cis-aconitic acid.

The invention also provides a method for reversibly inactivating a thermostable enzyme, comprising reacting a zero-length cross-linker compound with a carboxylic acid modifier reagent of the formula: 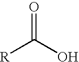
wherein R is a hydrogen, a substituted or unsubstituted phenyl group, a substituted or unsubstituted cycloalkyl group, a substituted or unsubstituted heteroaromatic group, or a substituted or unsubstituted alkyl group, to produce an activated carboxylic acid modifier reagent; and reacting said activated carboxylic acid modifier reagent with a thermostable enzyme to reversibly inactivate the thermostable enzyme. In further embodiments, the carboxylic acid modifier reagent is citraconic acid or cis-aconitic acid.

In some embodiments, the zero-length cross-linker provides an ester with the carboxylic acid modifier reagent. In certain embodiments, the zero-length cross-linker compound is a carbodiimide compound, Woodward's Reagent K, N,N′-Carbonyl Diimidazole, TSTU (O-(N-succinimidyl)-N,N,N′,N′-tetramethyluronium tetrafluoroborate), BTU (O-benzotriazol-1-yl)-N,N,N′,N′-tetramethyluronium hexafluorophosphate), TBTU (2-(1H-benzotriazo-1-yl)-1,1,3,3-tetramethyluronium hexafluorophosphate), TFFH (N,N′,N″,N′″-tetramethyluronium 2-fluoro-hexafluorophosphate), PyBOP (benzotriazole-1-yl-oxy-tris-pyrrolidino-phosphonium hexafluorophosphate), EEDQ (2-ethoxy-1-ethoxycarbonyl-1,2-dihydro-quinoline), DIPCDI (diisopropylcarbodiimide), MSNT (1-(mesitylene-2sulfonyl)-3-nitro-1H-1,2,4-triazole), or a triisopropylbenzenesulfonyl chloride. In further embodiments, the carbodiimide compound is 1-ethyl-3-(3-dimethylaminopropyl)carbodiimide hydrochloride (EDC), 1-Cyclohexyl-3-(2-morpholinoethyl)carbodiimide (CMC), dicyclohexylcarbodiimide (DCC), or Diisopropyl carbodiimide (DIC).

In some embodiments, the thermostable enzyme is a thermostable polymerase, such as a thermostable DNA polymerase or a thermostable RNA polymerase, a thermostable RNase H, a thermostable DNA nuclease, such as a thermostable DNA endonuclease, a thermostable DNA ligase, thermostable reverse transcriptase, thermostable helicase, thermostable RecA, and the like. In certain embodiments, the thermostable enzyme is a thermostable polymerase. In further embodiments, the thermostable polymerase is a thermostable DNA polymerase. In other embodiments, the thermostable polymerase is a thermostable RNA polymerase. In still other embodiments, the thermostable enzyme is a thermostable DNA nuclease, such as a thermostable DNA endonuclease. In other embodiments, the thermostable enzyme is derived from Thermus acquaticus, Thermus thermophilus, Thermatoga maritime, Aeropyrum pernix, Aquifex aeolicus, Archaeglobus fulgidus, Bacillus caldotenax, Carboxydothermus hydrogenformans, Methanobacterium thermoautotrophicum ΔH, Methanococcus jannaschii, Methanothermus fervidus, Pyrobaculum islandicum, Pyrococcus endeavori, Pyrococcus furiosus, Pyrococcus horihoshii, Pyrococcus profundus, Pyrococcus woesei, Pyrodictium occultum, Sulfolobus acidocaldarius, Sulfolobus solfataricus, Thermoanaerobacter thermohydrosulfuricus, Thermococcus celer, Thermococcus fumicolans, Thermococcus gorgonarius, Thermococcus kodakaraensis KOD1, Thermococcus litoralis, Thermococcus peptonophilus, Thermococcus sp. 9N-7, Thermococcus sp. TY, Thermococcus stetteri, Thermococcus zilligii, Thermoplasma acidophilum, Thermus brokianus, Thermus caldophilus GK24, Thermus flavus, Thermus rubens, or a mutant thereof.

The invention also provides a method for primer extension, by producing a primer extension reaction mixture by combining: a sample comprising a target nucleic acid; a first primer complementary to the target nucleic acid; and a thermostable polymerase composition; and incubating said primer extension reaction mixture to a temperature greater than about 50° C. for a period of time sufficient to activate said thermostable DNA polymerase composition so that said polymerase produces primer extension products from said first primer and said target nucleic acid.

In some embodiments, the primer extension reaction mixture further comprises a second primer complementary to the target nucleic acid. In certain embodiments, the method is a method of amplifying said target nucleic acid. In some embodiments, the thermostable polymerase is a thermostable DNA polymerase. In other embodiments, the thermostable polymerase is a thermostable RNA polymerase. In further embodiments, the thermostable polymerase is derived from Thermus acquaticus, Thermus thermophilus, Thermatoga maritime, Aeropyrum pernix, Aquifex aeolicus, Archaeglobus fulgidus, Bacillus caldotenax, Carboxydothermus hydrogenformans, Methanobacterium thermoautotrophicum ΔH, Methanococcus jannaschii, Methanothermus fervidus, Pyrobaculum islandicum, Pyrococcus endeavori, Pyrococcus furiosus, Pyrococcus horihoshii, Pyrococcus profundus, Pyrococcus woesei, Pyrodictium occultum, Sulfolobus acidocaldarius, Sulfolobus solfataricus, Thermoanaerobacter thermohydrosulfuricus, Thermococcus celer, Thermococcus fumicolans, Thermococcus gorgonarius, Thermococcus kodakaraensis KOD1, Thermococcus litoralis, Thermococcus peptonophilus, Thermococcus sp. 9° N-7, Thermococcus sp.,TY, Thermococcus stetteri, Thermococcus zilligii, Thermoplasma acidophilum, Thermus brokianus, Thermus caldophilus GK24, Thermus flavus, Thermus rubens, or a mutant thereof.

The invention also provides a primer extension reaction mixture, comprising a first primer; nucleotides; and a thermostable enzyme composition. In some embodiments, the mixture further comprises a second primer. In some embodiments, the nucleotides are ribonucleotides. In other embodiments, the nucleotides are deoxyribonucleotides. In some embodiments, the thermostable polymerase is a thermostable DNA polymerase. In other embodiments, the thermostable polymerase is a thermostable RNA polymerase. In further embodiments, the thermostable polymerase is derived from Thermus acquaticus, Thermus thermophilus, Thermatoga maritime, Aeropyrum pernix, Aquifex aeolicus, Archaeglobus fulgidus, Bacillus caldotenax, Carboxydothermus hydrogenformans, Methanobacterium thermoautotrophicum ΔH, Methanococcus jannaschii, Methanothermus fervidus, Pyrobaculum islandicum, Pyrococcus endeavori, Pyrococcus furiosus, Pyrococcus horihoshii, Pyrococcus profundus, Pyrococcus woesei, Pyrodictium occultum, Sulfolobus acidocaldarius, Sulfolobus solfataricus, Thermoanaerobacter thermohydrosulfuricus, Thermococcus celer, Thermococcus fumicolans, Thermococcus gorgonarius, Thermococcus kodakaraensis KOD1, Thermococcus litoralis, Thermococcus peptonophilus, Thermococcus sp. 9° N-7, Thermococcus sp. TY, Thermococcus stetteri, Thermococcus zilligii, Thermoplasma acidophilum, Thermus brokianus, Thermus caldophilus GK24, Thermus flavus, Thermus rubens, or a mutant thereof

The invention also provides a kit comprising a thermostable enzyme composition. In some embodiments, the thermostable enzyme is a thermostable polymerase, such as a thermostable DNA polymerase or a thermostable RNA polymerase, a thermostable RNase H, a thermostable DNA nuclease, such as a thermostable DNA endonuclease, a thermostable DNA ligase, thermostable reverse transcriptase, thermostable helicase, thermostable RecA, and the like. In certain embodiments, the thermostable enzyme is a thermostable polymerase. In further embodiments, the thermostable polymerase is a thermostable DNA polymerase. In other embodiments, the thermostable polymerase is a thermostable RNA polymerase. In still other embodiments, the thermostable enzyme is a thermostable DNA nuclease, such as a thermostable DNA endonuclease. In other embodiments, the thermostable enzyme is derived from Thermus acquaticus, Thermus thermophilus, Thermatoga maritime, Aeropyrum pernix, Aquifex aeolicus, Archaeglobus fulgidus, Bacillus caldotenax, Carboxydothermus hydrogenformans, Methanobacterium thermoautotrophicum ΔH, Methanococcus jannaschii, Methanothermus fervidus, Pyrobaculum islandicum, Pyrococcus endeavori, Pyrococcus furiosus, Pyrococcus horihoshii, Pyrococcus profundus, Pyrococcus woesei, Pyrodictium occultum, Sulfolobus acidocaldarius, Sulfolobus solfataricus, Thermoanaerobacter thermohydrosulfuricus, Thermococcus celer, Thermococcus fumicolans, Thermococcus gorgonarius, Thermococcus kodakaraensis KOD1, Thermococcus litoralis, Thermococcus peptonophilus, Thermococcus sp. 9° N-7, Thermococcus sp. TY, Thermococcus stetteri, Thermococcus zilligii, Thermoplasma acidophilum, Thermus brokianus, Thermus caldophilus GK24, Thermus flavus, Thermus rubens, or a mutant thereof.

These and other objects, advantages, and features of the invention will become apparent to those persons skilled in the art upon reading the details of the invention as more fully described below.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIG. 1 is a graph showing the results of an activity assay for modified Afu Flap endonuclease-1 (FEN-1). The results show that prior to activation, Afu FEN-1 did not display observable endonuclease activity. The X-axis shows the cycle number. Each cycle lasts 30 seconds. The Y-axis is the signal intensity for 6FAM. When Afu FEN-1 is active, the 6Fam probe is cleaved. Consequently, quenching of 6FAM by BHQ1 is released. If the enzyme is absent or completely inactive, 6FAM signal should remain flat.

FIG. 2 is a graph showing that incubation at 95° C. partially restores endonuclease activity of the chemically modified Afu FEN-1. The X-axis shows the cycle number. Each cycle lasts 30 seconds. The Y-axis is the signal intensity for 6FAM.

FIG. 3 is a graph showing a comparison of citraconic acid and cis-aconitic acid modified Afu FEN-1. The graph shows that both cis-aconitic acid modified enzyme as well as citraconic acid modified enzyme did not have any significant endonuclease activity. The X-axis shows the cycle number. Each cycle lasts 30 seconds. The Y-axis is the signal intensity for 6FAM.

FIG. 4 is a graph showing that both cis-aconitic acid modified enzyme as well as citraconic acid modified enzyme can be activated by incubation at 95° C. for 10 minutes. As shown, endonuclease activity of the citraconic acid modified Afu FEN-1 was restored 60˜70% more than the cis-aconitic acid modified Afu FEN-1. The X-axis shows the cycle number. Each cycle lasts 30 seconds. The Y-axis is the signal intensity for 6FAM.

FIG. 5 is a graph showing amplification with unmodified enzyme at pH 8.0 and at pH 8.7. The results show that neither cycle threshold (Ct) nor ΔRn were significantly affected by pH. The X-axis is PCR cycle number and the Y-axis shows the increase of SYBR® Green fluorescent dye signal intensity. SYBR® Green fluorescent dye stains double stranded DNA specifically and upon successful amplification of target nucleic acid, more double stranded DNA is made, resulting in the amplification of the signal.

FIG. 6 is a graph showing amplification with modified Taq DNA polymerase at pH 8.0 and at pH 8.7. In contrast to unmodified Taq DNA polymerase, amplification with modified Taq DNA polymerase was greatly impacted by pH. For example, Ct with the pH 8.7 system shifted nearly 10 cycles higher than with a pH 8.0 system. The X-axis is PCR cycle number and the Y-axis shows the increase of SYBR® Green fluorescent dye signal intensity.

FIG. 7 is a graph showing amplification of a target nucleic acid with DNA polymerase and 6 ng of either unmodified Afu FEN-1 endonuclease or reversibly chemically modified Afu FEN-1 endonuclease. The results show that while PCR with 6 ng of unmodified Afu FEN-1 was successful in detecting the target nucleic acid, the reaction produced a significantly weaker signal than the reaction containing the reversibly modified endonuclease. The X-axis shows the cycle number and the Y-axis is the signal intensity for 6FAM.

FIG. 8 is a is a graph showing amplification of a target nucleic acid with DNA polymerase and 10 ng of either unmodified Afu FEN-1 endonuclease or reversibly chemically modified Afu FEN-1 endonuclease. The results show that unlike 10 ng of unmodified Afu FEN-1 that totally failed to detect the target nucleic acid, detection with 10 ng modified Afu FEN-1 was successful. The X-axis shows the cycle number and the Y-axis is the signal intensity for 6FAM.

FIG. 9 is a graph showing the comparison between amplification of Target 3 (see Table 6) using modified DNA polymerase of the present invention (denoted as c. acid modified) and anhydride modified thermostable DNA polymerase under the Fast thermocycling conditions described in the Examples section. The X-axis is PCR cycle number and the Y-axis shows the increase of fluorescent dye signal intensity. Multiples lines for each enzyme type indicate replicate experiments.

FIG. 10 is a graph showing the comparison between amplification of Target 5 (see Table 6) using modified DNA polymerase of the present invention (denoted as c. acid modified) and anhydride modified thermostable DNA polymerase under the Fast thermocycling conditions described in the Examples section. The X-axis is PCR cycle number and the Y-axis shows the increase of fluorescent dye signal intensity. Multiples lines for each enzyme type indicate replicate experiments.

FIG. 11 is a graph showing the comparison between amplification of Target 8 (see Table 6) using modified DNA polymerase of the present invention (denoted as c. acid modified) and anhydride modified thermostable DNA polymerase under the Fast thermocycling conditions described in the Examples section. The X-axis is PCR cycle number and the Y-axis shows the increase of fluorescent dye signal intensity. Multiples lines for each enzyme type indicate replicate experiments.

FIG. 12 is an exemplary reaction scheme for modification of a thermostable enzyme with an active ester formed with a carboxylic acid and 1-ethyl-3-(3-dimethylaminopropyl)carbodiimide hydrochloride (EDC), a water soluble carbodiimide. Mixing of carboxylic acid and EDC results in the active ester o-acylisourea. When this active ester is added to an enzyme composition comprising the thermostable enzyme, the thermostable enzyme is modified primarily through amide bond formation through ε-amine of lysine residues on the enzyme. Heating of the modified enzyme causes hydrolysis of the amide bond and activation of the enzyme.

FIG. 13 is an exemplary reaction scheme for modification of a thermostable enzyme with an active ester formed with a carboxylic acid and N,N′-dicyclohexyl carbodiimide (DCC). DCC is a carbodiimide soluble in water and organic solvents. Mixing of carboxylic acid and DCC results in formation of the active ester o-acylisourea. When this active ester is added to an enzyme composition comprising the thermostable enzyme, the thermostable enzyme is modified primarily through amide bond formation through ε-amine of lysine residues on the enzyme. Heating of the modified enzyme causes hydrolysis of the amide bond and activation of the enzyme.

FIG. 14 is an exemplary reaction scheme for modification of a thermostable enzyme with an active ester formed with a carboxylic acid and N-ethyl-3-phenylisoxazolium-3′-sulfonate (Woodward's reagent K). Woodward's reagent K converts to a reactive ketoketenimine under alkaline condition. This reactive intermediate forms an enol ester with a carboxylic acid. When this enol ester is added to an enzyme solution, it is highly susceptible to nucleophilic attack. Reaction of the enol ester with an amine group, such as 6-amine of lysine residue on a thermostable enzyme, forms an amide bond. Heating of the modified enzyme causes hydrolysis of the amide bond and activation of the enzyme.

FIG. 15 is an exemplary reaction scheme for modification of a thermostable enzyme with an N-acylimidazole. The N-acylimidazole is formed by reaction of a carboxylic acid with a N,N′-carbonyldiimidazole (CDI). The yield of N-acylimidazole from the reaction is high due to the release of carbodioxide and imidazole. The N-acylimidazole is highly reactive with amine groups of the thermostable enzyme to form an amide bond in a properly buffered aqueous solution. Heating of the modified enzyme causes hydrolysis of the amide bond and activation of the enzyme.

FIG. 16 is an exemplary reaction scheme for modification of a thermostable enzyme with an N-hydroxysulfosuccinimide (Sulfo-NHS) ester. The sulfo-NHS ester is formed with a carboxylic acid, a carbodiimide and sulfo-NHS. Mixing of the carboxylic acid and EDC results in the active ester o-acylisourea. This active ester further reacts with sulfo-NHS to form a more stable sulfo-NHS ester. When the active sulfo-NHS ester is added to an enzyme composition comprising the thermostable enzyme, the thermostable enzyme is modified primarily through amide bond formation through ε-amine of lysine residues of the enzyme. Heating of the modified enzyme causes hydrolysis of the amide bond and activation of the enzyme.

FIG. 17 is an exemplary reaction scheme for modification of an enzyme with an N-hydroxysuccinimide (NHS) ester. The NHS ester is formed with a carboxylic acid, a carbodiimide and NHS. Mixing of carboxylic acid and EDC results in the active ester o-acylisourea. This active ester further reacts with NHS to form a more stable NHS ester. When the active NHS ester is added to a composition comprising the thermostable enzyme, the thermostable enzyme is modified primarily through amide bond formation through ε-amine of lysine residues of the enzyme. Heating of the modified enzyme causes hydrolysis of the amide bond and activation of the enzyme.

FIG. 18 shows possible side reactions when DCC is used as a zero-length cross-linker. In particular, spontaneous rearrangement of O-acylisourea to N-acylisourea occurs. While the O-acylisourea form is active, the N-acylisourea is not active.

FIG. 19 shows a second possible side reaction when DCC is used as a zero-length cross-linker. In particular, azlactone formation in the presence of an amino acid can occur. Although the azlactone reacts with amine group, it does not function as a zero-length cross-linker. Instead, ring opening amide bond formation produces a different molecule.

### DEFINITIONS

The terms “polynucleotide,” “oligonucleotide,” “nucleic acid” and “nucleic acid molecule” are used interchangeably herein to include a polymeric form of nucleotides, either ribonucleotides or deoxyribonucleotides. This term refers only to the primary structure of the molecule. Thus, the terms include triple-, double- and single-stranded DNA, as well as triple-, double- and single-stranded RNA. It also includes modifications, such as by methylation and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), any other type of polynucleotide which is an N- or C-glycoside of a purine or pyrimidine base, and other polymers containing nonnucleotidic backbones, for example, polyamide (e.g., peptide nucleic acids (PNAs)) and polymorpholino (commercially available from the Anti-Virals, Inc., Corvallis, Oreg., as Neugene) polymers, and other synthetic sequence-specific nucleic acid polymers providing that the polymers contain nucleobases in a configuration which allows for base pairing and base stacking, such as is found in DNA and RNA.

Unless specifically indicated otherwise, there is no intended distinction in length between the terms “polynucleotide,” “oligonucleotide,” “nucleic acid” and “nucleic acid molecule” and these terms will be used interchangeably. These terms refer only to the primary structure of the molecule. Thus, these terms include, for example, 3′-deoxy-2′,5′-DNA, oligodeoxyribonucleotide N3′P5′ phosphoramidates, 2′-O-alkyl-substituted RNA, double- and single-stranded DNA, as well as double- and single-stranded RNA, DNA:RNA hybrids, and hybrids between PNAs and DNA or RNA, and also include known types of modifications, for example, labels which are known in the art, methylation, “caps,” substitution of one or more of the naturally occurring nucleotides with an analog, internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters), those containing pendant moieties, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide. In particular, DNA is deoxyribonucleic acid.

Throughout the specification, abbreviations are used to refer to nucleotides (also referred to as bases), including abbreviations that refer to multiple nucleotides. As used herein, G=guanine, A=adenine, T=thymine, C=cytosine, and U=uracil. In addition, R=a purine nucleotide (A or G); Y=a pyrimidine nucleotide (C or T (U)); S=C or G; W=A or T(U); M=A or C; K=G or T(U); V=A, C or G; and N=any nucleotide (A, T(U), C, or G). Nucleotides can be referred to throughout using lower or upper case letters. It is also understood that nucleotide sequences provided for DNA in the specification also represent nucleotide sequences for RNA, where T is substituted by U.

The terms “deoxyribonucleic acid” and “DNA” as used herein mean a polymer composed of deoxyribonucleotides.

The terms “ribonucleic acid” and “RNA” as used herein refer to a polymer composed of ribonucleotides. Where sequences of a nucleic acid are provided using nucleotides of a DNA sequence, it is understood that such sequences encompass complementary DNA sequences and further also encompass RNA sequences based on the given DNA sequence or its complement, where uracil (U) replaces thymine (T) in the DNA sequence or its complement.

Two nucleotide sequences are “complementary” to one another when those molecules share base pair organization homology. “Complementary” nucleotide sequences will combine with specificity to form a stable duplex under appropriate hybridization conditions. For instance, two sequences are complementary when a section of a first sequence can bind to a section of a second sequence in an anti-parallel sense wherein the 3′-end of each sequence binds to the 5′-end of the other sequence and each A, T(U), G, and C of one sequence is then aligned with a T(U), A, C, and G, respectively, of the other sequence. RNA sequences can also include complementary G=U or U=G base pairs. Thus, two sequences need not have perfect homology to be “complementary” under the invention. Usually two sequences are sufficiently complementary when at least about 85% (preferably at least about 90%, and most preferably at least about 95%) of the nucleotides share base pair organization over a defined length of the molecule.

As used herein the term “isolated,” when used in the context of an isolated compound, refers to a compound of interest that is in an environment different from that in which the compound naturally occurs. “Isolated” is meant to include compounds that are within samples that are substantially enriched for the compound of interest and/or in which the compound of interest is partially or substantially purified. The term “isolated” encompasses instances in which the recited material is unaccompanied by at least some of the material with which it is normally associated in its natural state, preferably constituting at least about 0.5%, more preferably at least about 5% by weight of the total protein in a given sample. For example, the term “isolated” with respect to a polynucleotide generally refers to a nucleic acid molecule devoid, in whole or part, of sequences normally associated with it in nature; or a sequence, as it exists in nature, but having heterologous sequences in association therewith; or a molecule disassociated from the chromosome.

“Purified” as used herein means that the recited material comprises at least about 75% by weight of the total protein, with at least about 80% being preferred, and at least about 90% being particularly preferred. As used herein, the term “substantially pure” refers to a compound that is removed from its natural environment and is at least 60% free, preferably 75% free, and most preferably 90% free from other components with which it is naturally associated.

A polynucleotide “derived from” or “specific for” a designated sequence, such as a target sequence of a target nucleic acid, refers to a polynucleotide sequence which comprises a contiguous sequence of approximately at least about 6 nucleotides, preferably at least about 8 nucleotides, more preferably at least about 10-12 nucleotides, and even more preferably at least about 15-20 nucleotides corresponding to, i.e., identical or complementary to, a region of the designated nucleotide sequence. The derived polynucleotide will not necessarily be derived physically from the nucleotide sequence of interest, but may be generated in any manner, including, but not limited to, chemical synthesis, replication, reverse transcription or transcription, which is based on the information provided by the sequence of bases in the region(s) from which the polynucleotide is derived or specific for. Polynucleotides that are derived from” or “specific for” a designated sequence include polynucleotides that are in a sense or an antisense orientations relative to the original polynucleotide.

“Recombinant” as used herein to describe a nucleic acid molecule refers to a polynucleotide of genomic, cDNA, mammalian, bacterial, viral, semisynthetic, synthetic or other origin which, by virtue of its origin, manipulation, or both is not associated with all or a portion of the polynucleotide with which it is associated in nature. The term “recombinant” as used with respect to a protein or polypeptide means a polypeptide produced by expression of a recombinant polynucleotide.

A “DNA-dependent DNA polymerase” is an enzyme that synthesizes a complementary DNA copy from a DNA template. Examples include DNA polymerase I from E. coli and bacteriophage T7 DNA polymerase. All known DNA-dependent DNA polymerases require a complementary primer to initiate synthesis. Under suitable conditions, a DNA-dependent DNA polymerase may synthesize a complementary DNA copy from an RNA template.

A “DNA-dependent RNA polymerase” or a “transcriptase” is an enzyme that synthesizes multiple RNA copies from a double-stranded or partially-double stranded DNA molecule having a (usually double-stranded) promoter sequence. The RNA molecules (“transcripts”) are synthesized in the 5′ to 3′ direction beginning at a specific position just downstream of the promoter. Examples of transcriptases are the DNA-dependent RNA polymerase from E. coli and bacteriophages T7, T3, and SP6.

An “RNA-dependent DNA polymerase” or “reverse transcriptase” is an enzyme that synthesizes a complementary DNA copy from an RNA template. All known reverse transcriptases also have the ability to make a complementary DNA copy from a DNA template; thus, they are both RNA- and DNA-dependent DNA polymerases. A primer is required to initiate synthesis with both RNA and DNA templates.

“RNAse H” is an enzyme that degrades the RNA portion of an RNA:DNA duplex. These enzymes may be endonucleases or exonucleases. Most reverse transcriptase enzymes normally contain an RNAse H activity in addition to their polymerase activity. However, other sources of the RNAse H are available without an associated polymerase activity. RNA degradation mediated by an RNAse H may result in separation of RNA from a RNA:DNA complex, or the RNAse H may cut the RNA at various locations such that portions of the RNA melt off or permit enzymes to unwind portions of the RNA.

As used herein, the term “target nucleic acid region” or “target nucleic acid” or “target molecules” refers to a nucleic acid molecule with a “target sequence” to be detected (e.g., by amplification). The target nucleic acid may be either single-stranded or double-stranded and may or may not include other sequences besides the target sequence (e.g., the target nucleic acid may or may not include nucleic acid sequences upstream or 5′ flanking sequence, may or may not include downstream or 3′ flanking sequence, and in some embodiments may not include either upstream (5′) or downstream (3′) nucleic acid sequence relative to the target sequence. Where detection is by amplification, these other sequences in addition to the target sequence may or may not be amplified with the target sequence.

The term “target sequence” refers to the particular nucleotide sequence of the target nucleic acid to be detected (e.g., through amplification). The target sequence may include a probe-hybridizing region contained within the target molecule with which a probe will form a stable hybrid under desired conditions. The “target sequence” may also include the complexing sequences to which the oligonucleotide primers complex and can be extended using the target sequence as a template. Where the target nucleic acid is originally single-stranded, the term “target sequence” also refers to the sequence complementary to the “target sequence” as present in the target nucleic acid. If the “target nucleic acid” is originally double-stranded, the term “target sequence” refers to both the plus (+) and minus (−) strands. Moreover, where sequences of a “target sequence” are provided herein, it is understood that the sequence may be either DNA or RNA. Thus where a DNA sequence is provided, the RNA sequence is also contemplated and is readily provided by substituting “T” of the DNA sequence with “U” to provide the RNA sequence.

The term “primer” or “oligonucleotide primer” as used herein, refers to an oligonucleotide which acts to initiate synthesis of a complementary nucleic acid strand when placed under conditions in which synthesis of a primer extension product is induced, e.g., in the presence of nucleotides and a polymerization-inducing agent such as a DNA or RNA polymerase and at suitable temperature, pH, metal concentration, and salt concentration. Primers are generally of a length compatible with its use in synthesis of primer extension products, and are usually are in the range of between 8 to 100 nucleotides in length, such as 10 to 75, 15 to 60, 15 to 40, 18 to 30, 20 to 40, 21 to 50, 22 to 45, 25 to 40, and so on, more typically in the range of between 18-40, 20-35, 21-30 nucleotides long, and any length between the stated ranges. Typical primers can be in the range of between 10-50 nucleotides long, such as 15-45, 18-40, 20-30, 21-25 and so on, and any length between the stated ranges. In some embodiments, the primers are usually not more than about 10, 12, 15, 20, 21, 22, 23, 24, 25, 26, 27, 28, 29, 30, 35, 40, 45, 50, 55, 60, 65, or 70 nucleotides in length, more usually not more than about 10, 12, 15, 20, 21, 22, 23, 24, 25, 26, 27, 28, 29, or 30 nucleotides in length, still more usually not more than about 10, 12, 15, 20, 21, 22, 23, 24, or 25 nucleotides in length.

Primers are usually single-stranded for maximum efficiency in amplification, but may alternatively be double-stranded. If double-stranded, the primer is usually first treated to separate its strands before being used to prepare extension products. This denaturation step is typically effected by heat, but may alternatively be carried out using alkali, followed by neutralization. Thus, a “primer” is complementary to a template, and complexes by hydrogen bonding or hybridization with the template to give a primer/template complex for initiation of synthesis by a polymerase, which is extended by the addition of covalently bonded bases linked at its 3′ end complementary to the template in the process of DNA synthesis.

A “primer pair” as used herein refers to first and second primers having nucleic acid sequence suitable for nucleic acid-based amplification of a target nucleic acid. Such primer pairs generally include a first primer having a sequence that is the same or similar to that of a first portion of a target nucleic acid, and a second primer having a sequence that is complementary to a second portion of a target nucleic acid to provide for amplification of the target nucleic acid or a fragment thereof. Reference to “first” and “second” primers herein is arbitrary, unless specifically indicated otherwise. For example, the first primer can be designed as a “forward primer” (which initiates nucleic acid synthesis from a 5′ end of the target nucleic acid) or as a “reverse primer” (which initiates nucleic acid synthesis from a 5′ end of the extension product produced from synthesis initiated from the forward primer). Likewise, the second primer can be designed as a forward primer or a reverse primer.

The term “primer extension” as used herein refers to both to the synthesis of DNA resulting from the polymerization of individual nucleoside triphosphates using a primer as a point of initiation, and to the joining of additional oligonucleotides to the primer to extend the primer. As used herein, the term “primer extension” is intended to encompass the ligation of two oligonucleotides to form a longer product which can then serve as a target in future amplification cycles. As used herein, the term “primer” is intended to encompass the oligonucleotides used in ligation-mediated amplification processes which are extended by the ligation of a second oligonucleotide which hybridizes at an adjacent position.

Primers can incorporate additional features which allow for the detection or immobilization of the primer but do not alter the basic property of the primer, that of acting as a point of initiation of DNA synthesis. For example, primers may contain an additional nucleic acid sequence at the 5′ end which does not hybridize to the target nucleic acid, but which facilitates cloning of the amplified product. The region of the primer which is sufficiently complementary to the template to hybridize is referred to herein as the hybridizing region.

The term “non-specific amplification” refers to the amplification of nucleic acid sequences other than the target sequence which results from primers hybridizing to sequences other than the target sequence and then serving as a substrate for primer extension. The hybridization of a primer to a non-target sequence is referred to as “non-specific hybridization”, and can occur during the lower temperature, reduced stringency pre-reaction conditions.

The term “reaction mixture” refers to a solution containing reagents necessary to carry out a given reaction. An “amplification reaction mixture”, which refers to a solution containing reagents necessary to carry out an amplification reaction, typically contains oligonucleotide primers and a DNA polymerase or ligase in a suitable buffer. A “PCR reaction mixture” typically contains oligonucleotide primers, a thermostable DNA polymerase, dNTP's, and a divalent metal cation in a suitable buffer. A reaction mixture is referred to as complete if it contains all reagents necessary to enable the reaction, and incomplete if it contains only a subset of the necessary reagents. It will be understood by one of skill in the art that reaction components are routinely stored as separate solutions, each containing a subset of the total components, for reasons of convenience, storage stability, and to allow for independent adjustment of the concentrations of the components depending on the application, and, furthermore, that reaction components are combined prior to the reaction to create a complete reaction mixture.

As used herein, the term “probe” or “oligonucleotide probe”, used interchangeable herein, refers to a structure comprised of a polynucleotide, as defined above, which contains a nucleic acid sequence complementary to a nucleic acid sequence present in the target nucleic acid analyte (e.g., a nucleic acid amplification product). The polynucleotide regions of probes may be composed of DNA, and/or RNA, and/or synthetic nucleotide analogs. Probes are generally of a length compatible with its use in specific detection of all or a portion of a target sequence of a target nucleic acid, and are usually are in the range of between 8 to 100 nucleotides in length, such as 8 to 75, 10 to 74, 12 to 72, 15 to 60, 15 to 40, 18 to 30, 20 to 40, 21 to 50, 22 to 45, 25 to 40, and so on, more typically in the range of between 18-40, 20-35, 21-30 nucleotides long, and any length between the stated ranges. The typical probe is in the range of between 10-50 nucleotides long, such as 15-45, 18-40, 20-30, 21-28, 22-25 and so on, and any length between the stated ranges. In some embodiments, the probes are usually not more than about 10, 12, 15, 20, 21, 22, 23, 24, 25, 26, 27, 28, 29, 30, 35, 40, 45, 50, 55, 60, 65, or 70 nucleotides in length, more usually not more than about 10, 12, 15, 20, 21, 22, 23, 24, 25, 26, 27, 28, 29, 30, 35, or 40 nucleotides in length, still more usually not more than about 10, 12, 15, 20, 21, 22, 23, 24, or 25 nucleotides in length.

Probes contemplated herein include probes that include a detectable label. For example, when an “oligonucleotide probe” is to be used in a 5′ nuclease assay, such as the TaqMan™ assay, the probe includes at least one fluorescer and at least one quencher which is digested by the 5′ endonuclease activity of a polymerase used in the reaction in order to detect any amplified target oligonucleotide sequences. In this context, the oligonucleotide probe will have a sufficient number of phosphodiester linkages adjacent to its 5′ end so that the 5′ to 3′ nuclease activity employed can efficiently degrade the bound probe to separate the fluorescers acid quenchers. When an oligonucleotide probe is used in the TMA technique, it will be suitably labeled, as described below.

As used herein, the terms “label” and “detectable label” refer to a molecule capable of detection, including, but not limited to, radioactive isotopes, fluorescers, chemiluminescers, chromophores, enzymes, enzyme substrates, enzyme cofactors, enzyme inhibitors, chromophores, dyes, metal ions, metal sols, ligands (e.g., biotin, avidin, strepavidin or haptens) and the like. The term “fluorescer” refers to a substance or a portion thereof which is capable of exhibiting fluorescence in the detectable range.

The terms “hybridize” and “hybridization” refer to the formation of complexes between nucleotide sequences which are sufficiently complementary to form complexes via Watson-Crick base pairing. Where a primer “hybridizes” with target (template), such complexes (or hybrids) are sufficiently stable to serve the priming function required by, e.g., the DNA polymerase to initiate DNA synthesis.

The term “stringent conditions” refers to conditions under which a primer will hybridize preferentially to, or specifically bind to, its complementary binding partner, and to a lesser extent to, or not at all to, other sequences. Put another way, the term stringent hybridization conditions” as used herein refers to conditions that are compatible to produce duplexes between complementary binding members, e.g., between probes and complementary targets in a sample, e.g., duplexes of nucleic acid probes, such as DNA probes, and their corresponding nucleic acid targets that are present in the sample, e.g., their corresponding mRNA analytes present in the sample.

As used herein, the term “binding pair” refers to first and second molecules that specifically bind to each other, such as complementary polynucleotide pairs capable of forming nucleic acid duplexes. “Specific binding” of the first member of the binding pair to the second member of the binding pair in a sample is evidenced by the binding of the first member to the second member, or vice versa, with greater affinity and specificity than to other components in the sample. The binding between the members of the binding pair is typically noncovalent.

By “selectively bind” is meant that the molecule binds preferentially to the target of interest or binds with greater affinity to the target than to other molecules. For example, a DNA molecule will bind to a substantially complementary sequence and not to unrelated sequences.

A “stringent hybridization” and “stringent hybridization wash conditions” in the context of nucleic acid hybridization (e.g., as in array, Southern or Northern hybridizations) are sequence dependent, and are different under different environmental parameters. Stringent hybridization conditions that can be used to identify nucleic acids within the scope of the invention can include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1 % SDS at 65° C. Exemplary stringent hybridization conditions can also include a hybridization in a buffer of 40% formamide, 1 M NaCl, and 1% SDS at 37° C., and a wash in 1×SSC at 45° C. Alternatively, hybridization to filter-bound DNA in 0.5 M NaHPO₄, 7% sodium dodecyl sulfate (SDS), 1 mnM EDTA at 65° C., and washing in 0.1×SSC/0.1% SDS at 68° C. can be employed. Yet additional stringent hybridization conditions include hybridization at 60° C. or higher and 3×SSC (450 mM sodium chloride/45 mM sodium citrate) or incubation at 42° C. in a solution containing 30% formamide, 1M NaCl, 0.5% sodium sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions sets forth the conditions which determine whether a nucleic acid is specifically hybridized to a probe. Wash conditions used to identify nucleic acids may include, e.g.: a salt concentration of about 0.02 molar at pH 7 and a temperature of at least about 50.° C. or about 55° C. to about 60° C.; or, a salt concentration of about 0.15 M NaCl at 72° C. for about 15 minutes; or, a salt concentration of about 0.2×SSC at a temperature of at least about 50° C. or about 55. ° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C. In instances wherein the nucleic acid molecules are deoxyoligonucleotides (“oligos”), stringent conditions can include washing in 6×SSC/0.05% sodium pyrophosphate at 37.° C. (for 14-base oligos), 48.° C. (for 17-base oligos), 55° C. (for 20-base oligos), and 60° C. (for 23-base oligos). See Sambrook, Ausubel, or Tijssen (cited below) for detailed descriptions of equivalent hybridization and wash conditions and for reagents and buffers, e.g., SSC buffers and equivalent reagents and conditions.

Stringent hybridization conditions are hybridization conditions that are at least as stringent as the above representative conditions, where conditions are considered to be at least as stringent if they are at least about 80% as stringent, typically at least about 90% as stringent as the above specific stringent conditions. Other stringent hybridization conditions are known in the art and may also be employed, as appropriate.

The “melting temperature” or “Tm” of double-stranded DNA is defined as the temperature at which half of the helical structure of DNA is lost due to heating or other dissociation of the hydrogen bonding between base pairs, for example, by acid or alkali treatment, or the like. The Tₘ of a DNA molecule depends on its length and on its base composition. DNA molecules rich in GC base pairs have a higher Tₘ than those having an abundance of AT base pairs. Separated complementary strands of DNA spontaneously reassociate or anneal to form duplex DNA when the temperature is lowered below the Tₘ. The highest rate of nucleic acid hybridization occurs approximately 25° C. below the Tₘ. The Tₘ may be estimated using the following relationship: Tₘ=69.3+0.41(GC) % (Marmur et al. (1962) J. Mol. Biol. 5:109-118).

The term “organic group” and “organic radical” as used herein means any carbon-containing group, including hydrocarbon groups that are classified as an aliphatic group, cyclic group, aromatic group, functionalized derivatives thereof and/or various combination thereof. The term “aliphatic group” means a saturated or unsaturated linear or branched hydrocarbon group and encompasses alkyl, alkenyl, and alkynyl groups, for example. The term “alkyl group” means a substituted or unsubstituted, saturated linear or branched hydrocarbon group or chain (e.g., C₁ to C₈) including, for example, methyl, ethyl, isopropyl, tert-butyl, heptyl, iso-propyl, n-octyl, dodecyl, octadecyl, amyl, 2-ethylhexyl, and the like. Suitable substituents include carboxy, protected carboxy, amino, protected amino, halo, hydroxy, protected hydroxy, nitro, cyano, monosubstituted amino, protected monosubstituted amino, disubstituted amino, C₁ to C₇ alkoxy, C₁ to C₇ acyl, C₁ to C₇ acyloxy, and the like. The term “substituted alkyl” means the above defined alkyl group substituted from one to three times by a hydroxy, protected hydroxy, amino, protected amino, cyano, halo, trifloromethyl, mono-substituted amino, di-substituted amino, lower alkoxy, lower alkylthio, carboxy, protected carboxy, or a carboxy, amino, and/or hydroxy salt. As used in conjunction with the substituents for the heteroaryl rings, the terms “substituted (cycloalkyl)alkyl” and “substituted cycloalkyl” are as defined below substituted with the same groups as listed for a “substituted alkyl” group. The term “alkenyl group” means an unsaturated, linear or branched hydrocarbon group with one or more carbon-carbon double bonds, such as a vinyl group. The term “alkynyl group” means an unsaturated, linear or branched hydrocarbon group with one or more carbon-carbon triple bonds. The term “cyclic group” means a closed ring hydrocarbon group that is classified as an alicyclic group, aromatic group, or heterocyclic group. The term “alicyclic group” means a cyclic hydrocarbon group having properties resembling those of aliphatic groups. The term “aromatic group” or “aryl group” means a mono- or polycyclic aromatic hydrocarbon group, and may include one or more heteroatoms, and which are further defined below. The term “heterocyclic group” means a closed ring hydrocarbon in which one or more of the atoms in the ring are an element other than carbon (e.g., nitrogen, oxygen, sulfur, etc.), and are further defined below.

“Organic groups” may be functionalized or otherwise comprise additional functionalities associated with the organic group, such as carboxyl, amino, hydroxyl, and the like, which may be protected or unprotected. For example, the phrase “alkyl group” is intended to include not only pure open chain saturated hydrocarbon alkyl substituents, such as methyl, ethyl, propyl, t-butyl, and the like, but also alkyl substituents bearing further substituents known in the art, such as hydroxy, alkoxy, alkylsulfonyl, halogen atoms, cyano, nitro, amino, carboxyl, etc. Thus, “alkyl group” includes ethers, esters, haloalkyls, nitroalkyls, carboxyalkyls, hydroxyalkyls, sulfoalkyls, etc.

The terms “halo” and “halogen” refer to the fluoro, chloro, bromo or iodo groups. There can be one or more halogen, which are the same or different. Halogens of particular interest include chloro and bromo groups.

The term “haloalkyl” refers to an alkyl group as defined above that is substituted by one or more halogen atoms. The halogen atoms may be the same or different. The term “dihaloalkyl ” refers to an alkyl group as described above that is substituted by two halo groups, which may be the same or different. The term “trihaloalkyl” refers to an alkyl group as describe above that is substituted by three halo groups, which may be the same or different. The term “perhaloalkyl” refers to a haloalkyl group as defined above wherein each hydrogen atom in the alkyl group has been replaced by a halogen atom. The term “perfluoroalkyl” refers to a haloalkyl group as defined above wherein each hydrogen atom in the alkyl group has been replaced by a fluoro group.

The term “cycloalkyl” means a mono-, bi-, or tricyclic saturated ring that is fully saturated or partially unsaturated. Examples of such a group included cyclopropyl, cyclobutyl, cyclopentyl, cyclohexyl, cycloheptyl, adamantyl, cyclooctyl, cis- or trans decalin, bicyclo[2.2.1]hept-2-ene, cyclohex-1-enyl, cyclopent-1-enyl, 1,4-cyclooctadienyl, and the like.

The term “(cycloalkyl)alkyl” means the above-defined alkyl group substituted for one of the above cycloalkyl rings. Examples of such a group include (cyclohexyl)methyl, 3-(cyclopropyl)-n-propyl, 5-(cyclopentyl)hexyl, 6-(adamantyl)hexyl, and the like.

The term “substituted phenyl” specifies a phenyl group substituted with one or more moieties, and in some instances one, two, or three moieties, chosen from the groups consisting of halogen, hydroxy, protected hydroxy, cyano, nitro, trifluoromethyl, C₁ to C₇ alkyl, C₁ to C₇ alkoxy, C₁ to C₇ acyl, C₁ to C₇ acyloxy, carboxy, oxycarboxy, protected carboxy, carboxymethyl, protected carboxymethyl, hydroxymethyl, protected hydroxymethyl, amino, protected amino, (monosubstituted)amino, protected (monosubstituted)amino, (disubstituted)amino, carboxamide, protected carboxamide, N-(C₁ to C₆ alkyl)carboxamide, protected N-(C₁ to C₆ alkyl)carboxamide, N,N-di(C₁ to C₆ alkyl)carboxamide, trifluoromethyl, N-((C₁ to C₆ alkyl)sulfonyl)amino, N-(phenylsulfonyl)amino or phenyl, substituted or unsubstituted, such that, for example, a biphenyl or naphthyl group results.

Examples of the term “substituted phenyl” includes a mono- or di(halo)phenyl group such as 2, 3 or 4-chlorophenyl, 2,6-dichlorophenyl, 2,5-dichlorophenyl, 3,4-dichlorophenyl, 2, 3 or 4-bromophenyl, 3,4-dibromophenyl, 3-chloro-4-fluorophenyl, 2, 3 or 4-fluorophenyl and the like; a mono or di(hydroxy)phenyl group such as 2, 3, or 4-hydroxyphenyl, 2,4-dihydroxyphenyl, the protected-hydroxy derivatives thereof and the like; a nitrophenyl group such as 2, 3, or 4-nitrophenyl; a cyanophenyl group, for example, 2, 3 or 4-cyanophenyl; a mono- or di(alkyl)phenyl group such as 2, 3, or 4-methylphenyl, 2,4-dimethylphenyl, 2, 3 or 4-(iso-propyl)phenyl, 2, 3, or 4-ethylphenyl, 2, 3 or 4-(n-propyl)phenyl and the like; a mono or di(alkoxy)phenyl group, for example, 2,6-dimethoxyphenyl, 2, 3 or 4-(isopropoxy)phenyl, 2, 3 or 4-(t-butoxy)phenyl, 3-ethoxy-4-methoxyphenyl and the like; 2, 3 or 4-trifluoromethylphenyl; a mono- or dicarboxyphenyl or (protected carboxy)phenyl group such as 2, 3 or 4-carboxyphenyl or 2,4-di(protected carboxy)phenyl; a mono- or di(hydroxymethyl)phenyl or (protected hydroxymethyl)phenyl such as 2, 3 or 4-(protected hydroxymethyl)phenyl or 3,4-di(hydroxymethyl)phenyl; a mono- or di(aminomethyl)phenyl or (protected aminomethyl)phenyl such as 2, 3 or 4-(aminomethyl)phenyl or 2,4-(protected aminomethyl)phenyl; or a mono- or di(N-(methylsulfonylamino))phenyl such as 2, 3 or 4-(N-(methylsulfonylamino))phenyl. Also, the term “substituted phenyl” represents disubstituted phenyl groups wherein the substituents are different, for example, 3-methyl-4-hydroxyphenyl, 3-chloro-4-hydroxyphenyl, 2-methoxy-4-bromophenyl, 4-ethyl-2-hydroxyphenyl, 3-hydroxy-4-nitrophenyl, 2-hydroxy-4-chlorophenyl and the like.

The term “(substituted phenyl)alkyl” means one of the above substituted phenyl groups attached to one of the above-described alkyl groups. Examples of include such groups as 2-phenyl-1-chloroethyl, 2-(4′-methoxyphenyl)ethyl, 4-(2′,6′-dihydroxy phenyl)n-hexyl, 2-(5′-cyano-3′-methoxyphenyl)n-pentyl, 3-(2′,6′-dimethylphenyl)n-propyl, 4-chloro-3-aminobenzyl, 6-(4′-methoxyphenyl)-3-carboxy(n-hexyl), 5-(4′-aminomethylphenyl)-3-(aminomethyl)n-pentyl, 5-phenyl-3-oxo-n-pent-1-yl, (4-hydroxynapth-2-yl)methyl and the like.

As noted above, the term “aromatic” or “aryl” refers to six membered carbocyclic rings. Also as noted above, the term “heteroaryl” denotes optionally substituted five-membered or six-membered rings that have 1 to 4 heteroatoms, such as oxygen, sulfur and/or nitrogen atoms, in particular nitrogen, either alone or in conjunction with sulfur or oxygen ring atoms.

Furthermore, the above optionally substituted five-membered or six-membered rings can optionally be fused to an aromatic 5-membered or 6-membered ring system. For example, the rings can be optionally fused to an aromatic 5-membered or 6-membered ring system such as a pyridine or a triazole system, and preferably to a benzene ring.

The following ring systems are examples of the heterocyclic (whether substituted or unsubstituted) radicals denoted by the term “heteroaryl”: thienyl, furyl, pyrrolyl, pyrrolidinyl, imidazolyl, isoxazolyl, triazolyl, thiadiazolyl, oxadiazolyl, tetrazolyl, thiatriazolyl, oxatriazolyl, pyridyl, pyrimidyl, pyrazinyl, pyridazinyl, oxazinyl, triazinyl, thiadiazinyl tetrazolo, 1,5-[b]pyridazinyl and purinyl, as well as benzo-fused derivatives, for example, benzoxazolyl, benzthiazolyl, benzimidazolyl and indolyl.

Substituents for the above optionally substituted heteroaryl rings are from one to three halo, trihalomethyl, amino, protected amino, amino salts, mono-substituted amino, di-substituted amino, carboxy, protected carboxy, carboxylate salts, hydroxy, protected hydroxy, salts of a hydroxy group, lower alkoxy, lower alkylthio, alkyl, substituted alkyl, cycloalkyl, substituted cycloalkyl, (cycloalkyl)alkyl, substituted (cycloalkyl)alkyl, phenyl, substituted phenyl, phenylalkyl, and (substituted phenyl)alkyl. Substituents for the heteroaryl group are as heretofore defined, or in the case of trihalomethyl, can be trifluoromethyl, trichloromethyl, tribromomethyl, or triiodomethyl. As used in conjunction with the above substituents for heteroaryl rings, “lower alkoxy” means a C₁ to _{C}4 alkoxy group, similarly, “lower alkylthio” means a C₁ to C₄ alkylthio group.

The term “(monosubstituted)amino” refers to an amino group with one substituent chosen from the group consisting of phenyl, substituted phenyl, alkyl, substituted alkyl, C₁ to C₄ acyl, C₂ to C₇ alkenyl, C₂ to C₇ substituted alkenyl, C₂ to C₇ alkynyl, C₇ to C₁₆ alkylaryl, C₇ to C₁₆ substituted alkylaryl and heteroaryl group. The (monosubstituted) amino can additionally have an amino-protecting group as encompassed by the term “protected (monosubstituted)amino.” The term “(disubstituted)amino” refers to amino groups with two substituents chosen from the group consisting of phenyl, substituted phenyl, alkyl, substituted alkyl, C₁ to C₇ acyl, C₂ to C₇ alkenyl, C₂ to C₇ alkynyl, C₇ to C₁₆ alkylaryl, C₇ to C₁₆ substituted alkylaryl and heteroaryl. The two substituents can be the same or different.

The term “heteroaryl(alkyl)” denotes an alkyl group as defined above, substituted at any position by a heteroaryl group, as above defined.

The term “assessing” includes any form of measurement, and includes determining if an element is present or not. The terms “determining”, “measuring”, “evaluating”, “assessing” and “assaying” are used interchangeably and includes quantitative and qualitative determinations. Assessing may be relative or absolute. “Assessing the presence of′ includes determining the amount of something present, and/or determining whether it is present or absent. As used herein, the terms “determining,” “measuring,” and “assessing,” and “assaying” are used interchangeably and include both quantitative and qualitative determinations.

“Precision” refers to the ability of an assay to reproducibly generate the same or comparable result for a given sample.

“Accuracy” refers to the ability of an assay to correctly detect a target molecule in a blinded panel containing both positive and negative specimens.

### DETAILED DESCRIPTION OF THE INVENTION

The present invention provides reversibly modified thermostable enzyme compositions. Also provided are methods of making the subject compositions, e.g., by modifying a thermostable enzyme with a carboxylic acid modifier reagent. The present invention also provides methods of using the reversibly modified thermostable enzyme compositions, as well as kits and systems comprising the reversibly modified thermostable enzyme compositions.

Before the present invention is described further, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a enzyme” includes a plurality of such enzymes and reference to “the primer” includes reference to one or more primers and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Reversibly Inactivated Thermostable Enzyme Compositions

As noted above, the present invention provides reversibly modified thermostable enzyme compositions. As used herein, the term “thermostable enzyme” refers to an enzyme that is relatively stable to heat. The thermostable enzymes can withstand the high temperature incubation used to remove the modifier groups, typically greater than 50° C., without suffering an irreversible loss of activity. Modified thermostable enzymes usable in the methods of the present invention include, for example, thermostable polymerase, such as a thermostable DNA polymerase or a thermostable RNA polymerase, a thermostable RNase H, a thermostable DNA nuclease, such as a thermostable DNA endonuclease, a thermostable DNA ligase, thermostable reverse transcriptase, thermostable helicase, thermostable RecA, and the like.

In some embodiments the thermostable enzyme is a thermostable DNA polymerase. The term “thermostable DNA polymerase” refers to an enzyme that is relatively stable to heat and catalyzes the polymerization of nucleoside triphosphates to form primer extension products that are complementary to one of the nucleic acid strands of the target sequence. The enzyme initiates synthesis at the 3′ end of the primer and proceeds in the direction toward the 5′ end of the template until synthesis terminates. Purified thermostable DNA polymerases are described in U.S. Pat. No. 4,889,818; U.S. Pat. No. 5,352,600; U.S. Pat. No. 5,079,352; PCT/US90/07639; PCT/US91/05753; PCT/US91/0703; PCT/US91/07076; co-pending U.S. patent application Ser. No. 08/062,368; WO 92/09689; and U.S. Pat. No. 5,210,036; each incorporated herein by reference.

In certain embodiments, the thermostable enzyme is derived from Thermus acquaticus, Thermus thermophilus, Thermatoga maritime, Aeropyrum pernix, Aquifex aeolicus, Archaeglobus fulgidus, Bacillus caldotenax, Carboxydothermus hydrogenformans, Methanobacterium thermoautotrophicum ΔH, Methanococcus jannaschii, Methanothermus fervidus, Pyrobaculum islandicum, Pyrococcus endeavori, Pyrococcus furiosus, Pyrococcus horihoshii, Pyrococcus profundus, Pyrococcus woesei, Pyrodictium occultum, Sulfolobus acidocaldarius, Sulfolobus solfataricus, Thermoanaerobacter thermohydrosulfuricus, Thermococcus celer, Thermococcus fumicolans, Thermococcus gorgonarius, Thermococcus kodakaraensis KOD1, Thermococcus litoralis, Thermococcus peptonophilus, Thermococcus sp. 9° N-7, Thermococcus sp. TY, Thermococcus stetteri, Thermococcus zilligii, Thermoplasma acidophilum, Thermus brokianus, Thermus caldophilus GK24, Thermus flavus, Thermus rubens, or a mutant thereof.

In certain embodiments, the thermostable enzyme is a thermostable nuclease, such as a thermostable DNA endonuclease. In further embodiments, the thermostable nuclease is a thermostable DNA nuclease derived from Archeoglobus fuldigus. The term “thermostable endonuclease” refers to an enzyme that is relatively stable to heat and catalyzes catalyzes the hydrolysis of phosphodiester bonds between nucleic acids in a DNA molecule or an RNA molecule.

As such, the present invention provides for enzyme compositions of a thermostable enzyme that has been reversibly inactivated. The term “reversibly inactivated”, as used herein, refers to an enzyme which has been inactivated by reaction with a compound which results in the covalent modification (also referred to as chemical modification) of the enzyme, wherein the modifier compound is removable under appropriate conditions.

A feature of the subject enzyme compositions is that incubation of the modified thermostable enzyme composition in an aqueous buffer at a temperature greater that about 50° C., including from about 55° C. to about 100° C., such as from about 60° C. to about 95° C., from about 65° C. to about 90° C., from about 70° C. to about 85° C., including a temperature greater than about 80° C. results in at least a two fold increase, including at least about a three fold increase, about a five fold increase, about a seven fold increase, about 10 fold increase, about fifteen fold increase, about a twenty fold increase or more in enzyme activity. The buffer may be formulated from about pH 7 to about pH 9. at 25° C., including from about pH 7.25 to about pH 8.75, from about pH 7.5 to about pH 8.8, from about pH 7.75 to about pH 8.25, and about pH 8.0.

In some embodiments, incubation of the modified thermostable enzyme composition in an aqueous buffer, formulated to about pH 7 to about pH 9 at 25° C., at a temperature greater that about 50° C. results in at least a two-fold increase in enzyme activity in less than about 20 minutes. In other embodiments, incubation of the modified thermostable enzyme composition in an aqueous buffer, formulated to about pH 7 to about pH 8 at 25° C., at a temperature greater that about 50° C. results in at least a two-fold increase in enzyme activity in less than about 20 minutes.

Methods of Making the Subject Enzyme Compositions

The subject compositions can be made using any convenient methods. In a representative embodiment, the compositions are produced by modifying an initial thermostable enzyme composition with a carboxylic modifying reagent under conditions sufficient to produce the desired enzyme compositions, as described above.

The reaction which results in the removal of the modifier compound need not be the reverse of the modification reaction. As long as there is a reaction which results in removal of the modifier compound and restoration of enzyme function, the enzyme is considered to be reversibly inactivated.

According to the present invention, a thermostable enzyme is modified with an activated carboxylic acid modifying reagent, wherein reaction of the reagent with the enzyme results in covalent attachment of at least one carboxylic acid group to at least one amine group, such as a ε-amine group of a lysine residue, of the thermostable enzyme. In certain embodiments, activation of carboxylic acid is done with a zero-length cross-linker alone or in combination with sulfo-NHS or NHS compound. A carboxylic acid suitable for use with the present invention can be any carboxylic acid that can be activated by a zero-length cross-linker alone or in combination with sulfo-NHS or NHS, and can form a covalent bond with the thermostable enzyme that results in inactivation of the thermostable enzyme.

Suitable carboxylic acid reagents comprise the following general formula: 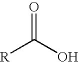
wherein R is a hydrogen, a substituted or unsubstituted phenyl group, a substituted or unsubstituted cycloalkyl group, a substituted or unsubstituted heteroaromatic group, or a substituted or unsubstituted alkyl group such as a substituted or unsubstituted, saturated linear or branched hydrocarbon group or chain (e.g., C₁ to C₈ ) including, e.g., methyl, ethyl, isopropyl, tert-butyl, heptyl, n-octyl, dodecyl, octadecyl, amyl, 2-ethylhexyl.

Exemplary carboxylic acid reagents include the following:

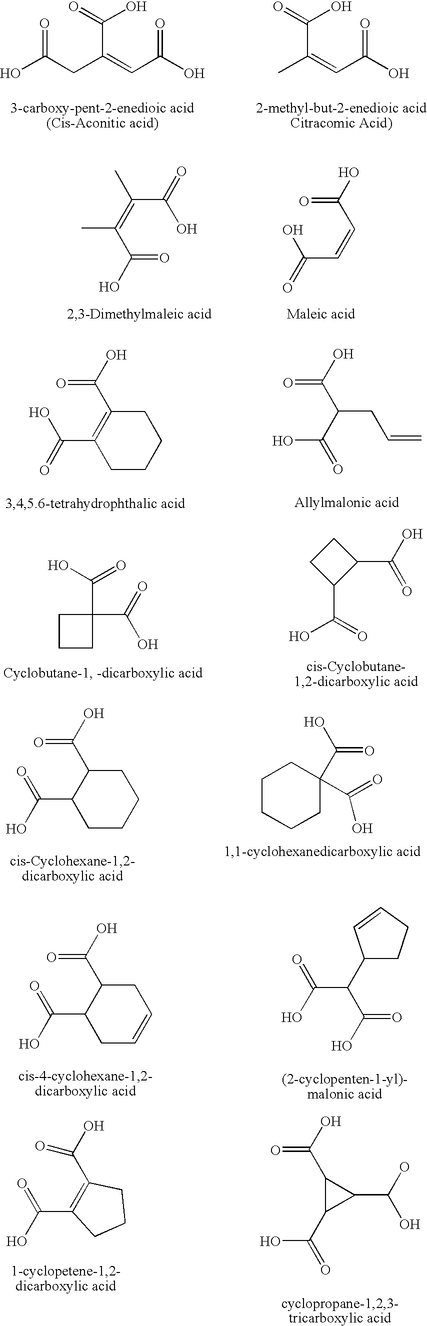

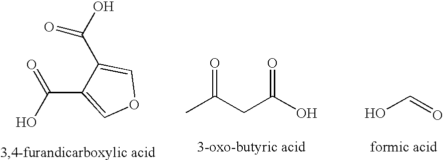

Selection of a carboxylic acid reagent for modification of any specific thermostable enzyme depends on the thermostability of the enzyme and the temperature requirement for the nucleic acid detection process. In particular, activation of the modified thermostable enzyme should not significantly harm other components involved in the reaction mixture such as template nucleic acid, dNTPs, NAD, or any other protein molecules present in the mixture for use in nucleic acid detection, such as carrier protein, e.g., BSA or gelatin, that may be used improve detection. The stability of the covalent bond formed between the carboxylic acid modifying reagent and the thermostable enzyme is dependant on the selection of the carboxylic acid reagent.

According to certain embodiments of the present invention, conjugation of the carboxylic acid reagent with a thermostable enzyme is mediated by a zero-length cross-linker. Activation of carboxylic acid is carried out with a zero-length cross-linker. Zero-length cross-linker refers to compounds mediating covalent bond formation between the carboxylic acid and the enzyme without adding additional atoms to the bond.

Suitable zero-length cross linkers react with carboxylic acids to form —C(O)R₁—OR₂, where R₁ is a good leaving group. Examples of good leaving groups are: oxysuccinimidyl; oxysulfosuccinimidyl; 1-oxybenzotriazolyl; and R₂ is selected from the group consisting of (C₄-C₂₀)aryl, cycloalkyl(e.g., cyclohexyl), heterocycloalkyl, (C₅-C₂₀)aryl, (C₅-C₂₀)aryl, (C₅-C₂₀)aryl substituted with one or more of the same or different electron withdrawing groups (e.g., —NO₂, —F, —Cl, —CN, —CF₃, etc.), heteroaryl, and heteroaryl substituted with one or more of the same or different electron withdrawing groups, n-dialkylaminoalkyls (e.g., 3-dimethylaminopropyl) and N-morpholinomethyl. Examples of suitable compounds include, but are not limited to a carbodiimide reagent, e.g. dicyclohexylcarbodiimide (DCC), diisopropylcarbodiimide (DIC), 1-ethyl-3-(3-dimethylaminopropyl)carbodiimide hydrochloride (EDC), 1-Cyclohexyl-3-(2-morpholinoethyl)carbodiimide (CMC), a uranium reagent, e.g. TSTU(O-(N-succinimidyl)-N,N,N′,N′-tetramethyluronium tetrafluoroborate), HBTU(O-benzotriazol-1-yl)-N,N,N′,N′-tetramethyluronium hexafluorophosphate), an activator, such as 1-hydroxybenzotriazole (HOBt), and N-hydroxysuccinimide to give NHS ester of the carboxylic acid; a carbodiimide with an NHS or sulfo-NHS; Woodward's Reagent K; N,N′-Carbonyl Diimidazole (CDI); TBTU (2-(1H-benzotriazo-1-yl)-1,1,3,3-tetramethyluronium hexafluorophosphate); TFFH (N,N′,N″,N′″-tetramethyluronium 2-fluoro-hexafluorophosphate); PyBOP (benzotriazole-1-yl-oxy-tris-pyrrolidino-phosphonium hexafluorophosphate); EEDQ (2-ethoxy-1-ethoxycarbonyl-1,2-dihydro-quinoline); DIPCDI (diisopropylcarbodiimide); MSNT( 1-(mesitylene-2sulfonyl)-3-nitro-1H-1,2,4-triazole); and aryl sulfonyl halides, e.g. triisopropylbenzenesulfonyl chloride.

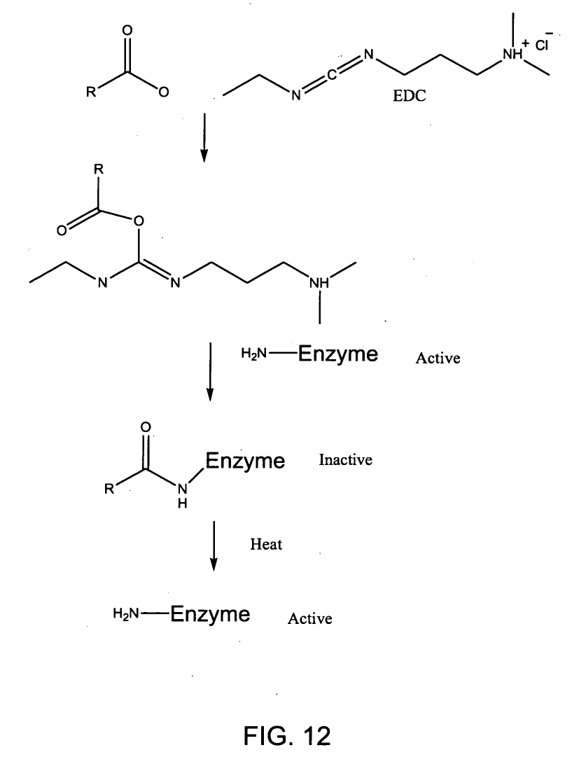

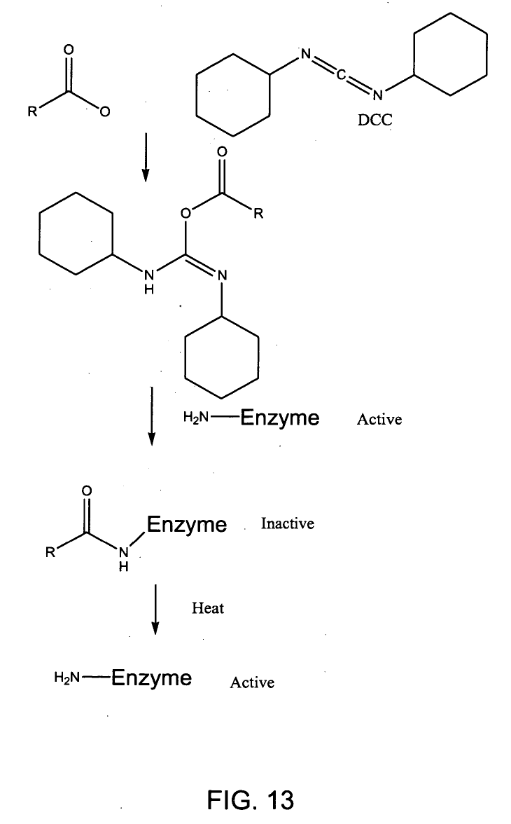

In one embodiment, the zero-length cross-linker is a carbodiimide, such as EDC, CMC, DCC, DIC. In further embodiments, the carboxylic acid reagent is cis-aconitic acid or citraconic acid. An exemplary reaction scheme using EDC is provided in FIG. 12. An exemplary reaction scheme using DCC is provided in FIG. 13. A carbodiimide forms an active ester with a carboxylic acid reagent. The active ester can then covalently attach to the thermostable enzyme molecule. The modification results in covalent attachment of at least one carboxylic acid group to at least one amine group, such as a ε-amine group of a lysine residue, of the thermostable enzyme. EDC and CMC are water-soluble while DCC is soluble both in water and organic solvents. DIC is water-insoluble but soluble in organic solvents. Because many carboxylic acids are both soluble in water and organic solvents, activation of carboxylic acid can be done in aqueous or organic solvents or aqueous/organic mixed solvents. All molecules and reaction products should be stable and not have significant side reactions, such as structural rearrangements. In certain embodiments, the activation is performed in an organic solvent, such as DMF, DMSO, acetone, dioxane, acetonitrile, THF, and the like, since the active ester formed in an aqueous solution may undergo hydrolysis,.

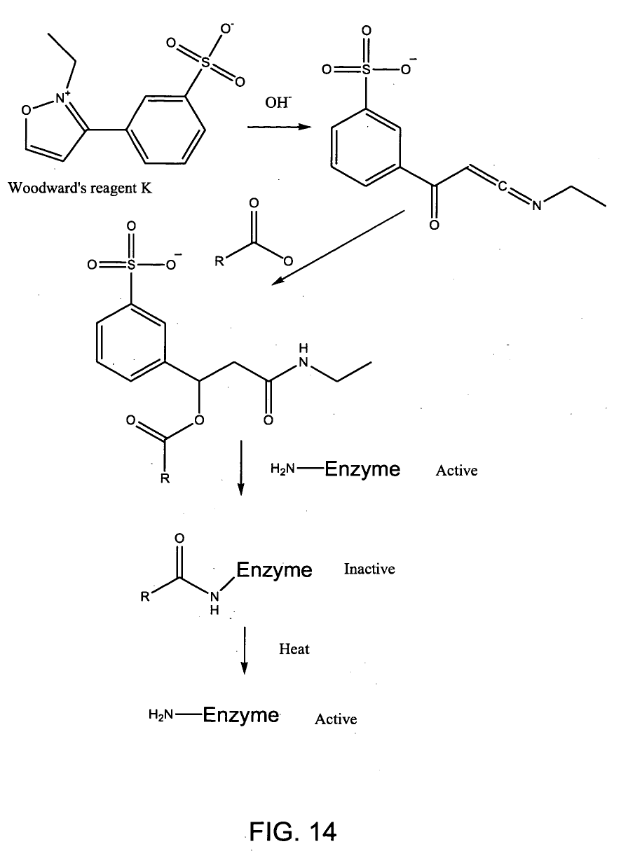

In another embodiment, the zero-length cross-linker is N-ethyl-3-phenylisoxazolium-3′-sulfonate (Woodward's reagent K). In a further embodiment, the carboxylic acid reagent is cis-aconitic acid or citraconic acid. An exemplary reaction scheme using Woodward's reagent K is provided in FIG. 14. Under alkaline condition, Woodward's reagent K is first converted to a reactive ketoketenimine that is then used to form an enol ester with a carboxylic acid reagent. The enol ester is highly susceptible to nucleophilic reaction. When a nucleophilic group is an amine group such as ε-amine group of lysine, an amide bond is formed as the result. Due to rapid hydrolysis of the enol ester, it is recommended to use freshly prepared enol ester for enzyme modification.

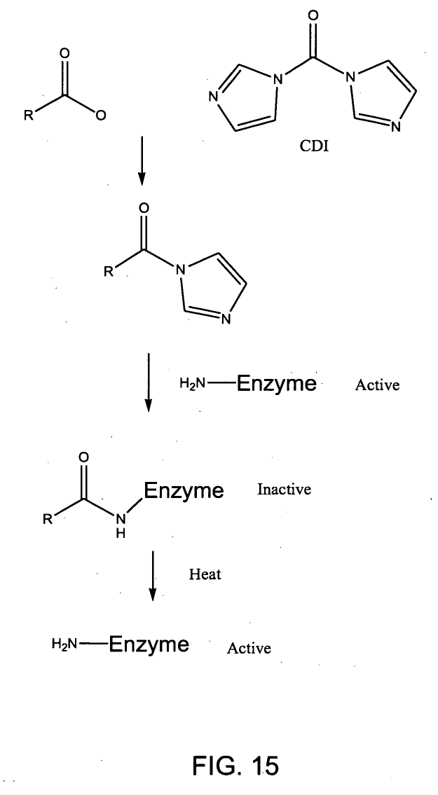

In yet another embodiment, the zero-length cross-linker is N,N′-carbonyldiimidazole (CDI). In a further embodiment, the carboxylic acid reagent is cis-aconitic acid or citraconic acid. An exemplary reaction scheme using CDI is provided in FIG. 15. CDI contains two acylimidazole groups and is a very reactive carbonylating agent. A carboxylic acid group reacts with CDI to form N-acylimidazoles, which are highly reactive with amine group. Release of carbon dioxide and imidazole makes the reaction irreversible resulting in a high yield. The imidazole in N-acylimidazole is released when an amine group attacks N-acylimidazole. As the result, an amide bond is formed. Activation of carboxylic acid with CDI should be performed in non-aqueous solvents because CDI hydrolyzes rapidly in water, even in a small percentage, to release carbon dioxide and imidazoles. Dry organic solvents are exemplary solvents for the activation reaction.

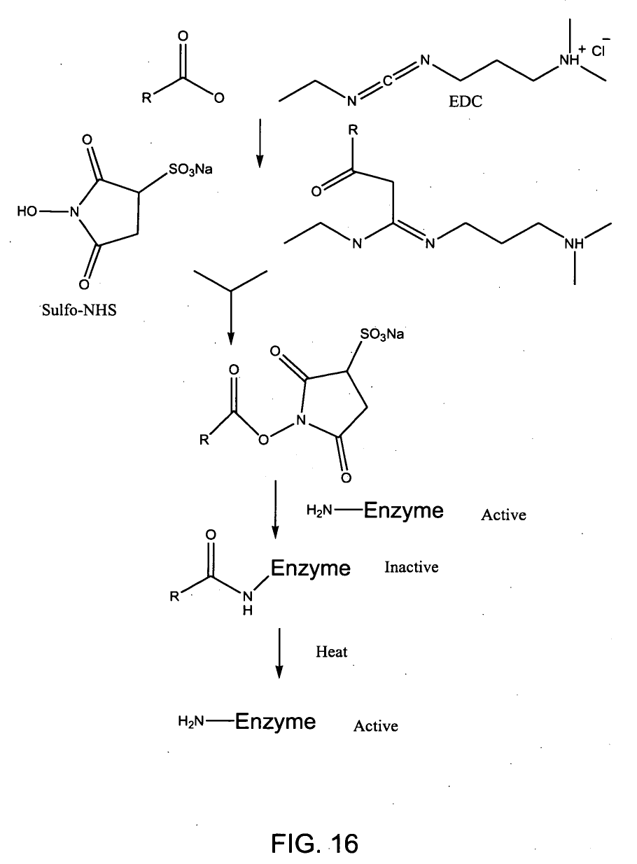

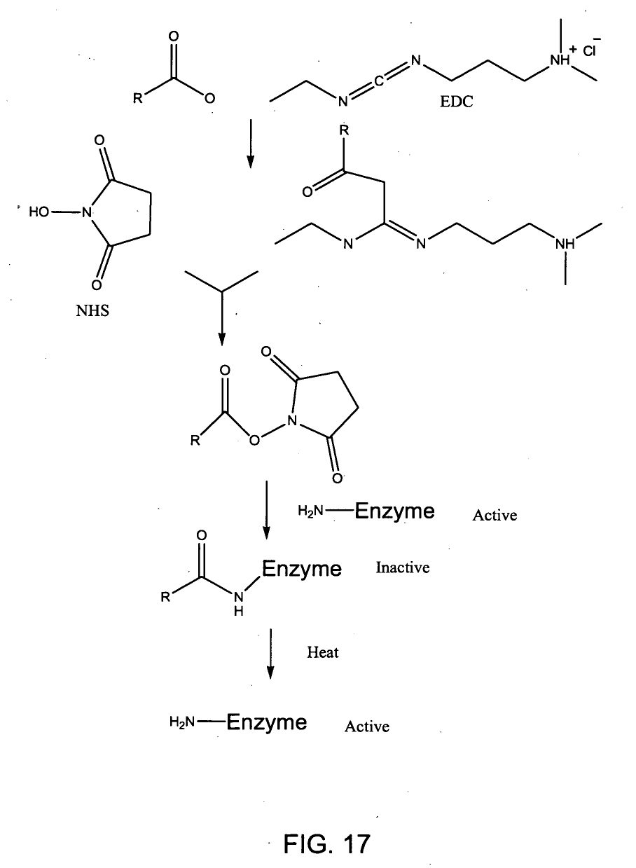

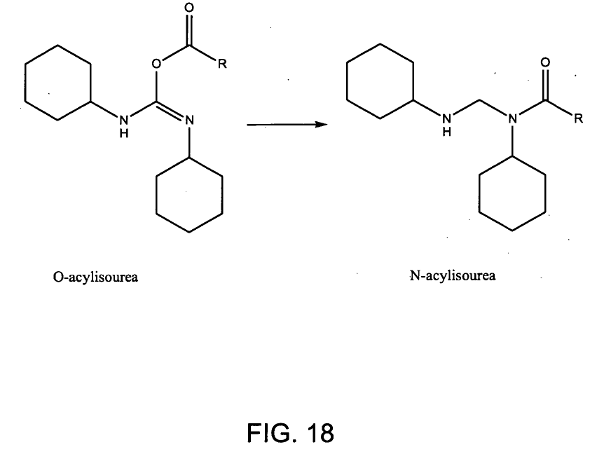

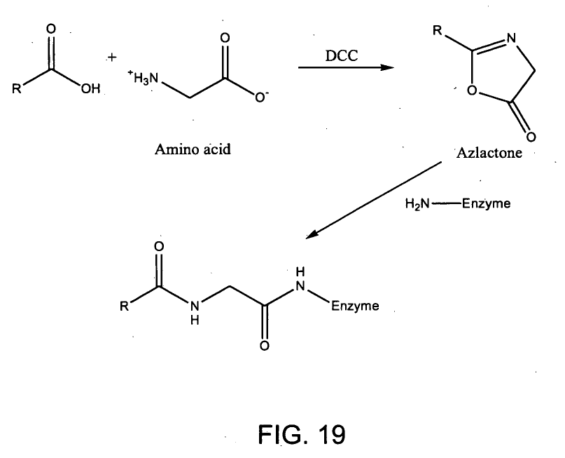

In some embodiments, activation of the carboxylic acid reagent is performed with a zero-length cross-linker and another molecule which can form an active molecule with higher stability under modification condition. In one embodiment, the second molecule is sulfo-NHS. An exemplary reaction scheme using sulfo-NHS is provided in FIG. 16. The use of a second compound, such as sulfo-NHS is that reaction results in less hydrolysis of the sulfo-NHS ester in aqueous solution and therefore reduced rearrangement of the sulfo-NHS ester. EDC is a widely used water-soluble zero-length cross-linker. It forms O-acylisourea, an active ester, with a carboxylic acid reagent. However, the O-acylisourea compound is not stable in an aqueous solution and hydrolyzes rapidly (Hoare, 1967, JBC, 242:2447-2453). The quick hydrolysis makes modification of enzyme less efficient. However, in the presence of a sulfo-NHS molecule, O-acylisourea reacts with sulfo-NHS to generate sulfo-NHS ester, a hydrophilic molecule which quickly reacts with amine groups (Staros et al., 1986, Anals. Biochem., 156:220-222). Sulfo-NHS ester hydrolyzes in water solution at a reduced rate. Its extraordinary stability in water makes it a very effective intermediate for enzyme modification in an aqueous environment. Besides its advantage in stability, sulfo-NHS ester does not have side reactions observed with some other active esters. DCC is one of the most frequently used coupling reagents. There are at least two side-reactions associated with DCC that have been reported: one is spontaneous rearrangement of active O-acylisourea to form an inactive N-acylisourea (FIG. 18); the other is formation of an azlactone which no longer functions as a zero-length cross-linker (FIG. 19). Another zero-length cross-linker, DIC, behaves in a similar way to DCC. All side-reactions occurred with DCC may happen to DIC as well. In contrast, no such problems are associated with use of sulfo-NHS ester.

In another embodiment, such a molecule is NHS. An exemplary reaction scheme using sulfo-NHS is provided in FIG. 16. The benefits of using NHS are essentially the same as sulfo-NHS. The primary difference is water-solubility. Sulfo-NHS and its esters have improved water solubility in comparison with NHS. If an active ester is not formed in the aqueous solution, sulfo-NHS can be replaced with NHS without significant impact on the modification process.

Modification of a thermostable enzyme can be performed in a one-step reaction, wherein carboxylic acid activation and modification of the thermostable enzyme happen simultaneously. In addition, the modification of the thermostable enzyme can be performed in a two-step process. The first step is activation of the carboxylic acid reagent and the second step is modification of the thermostable enzyme with pre-activated carboxylic acid. The first step can be carried out in an organic solvent to completely avoid hydrolysis of the zero-length cross-linker and pre-activated carboxylic acid reagent. In such a scheme, the yield of pre-activated carboxylic acid can be very high. In the absence of water molecules, the pre-activated carboxylic acid reagent can be stored for a long period of time without being broken down. The second step is the modification of the thermostable enzyme with the pre-activated carboxylic acid reagent. Because the activated carboxylic acid reagent is pre-formed, efficient modification can be achieved without using high concentrations of the reactants. This makes it possible to use zero-length cross-linkers having poor water solubility. It is also easier to control pH of the reaction system, which is critical for the modification reaction.

Utility

The subject enzyme compositions find use in a variety of different applications, representative applications being reviewed in greater detail below. In representative embodiments, the present invention provides methods of using the reversibly modified thermostable enzymes for nucleic acid detection, such as primer extension, by contacting a sample comprising a target nucleic acid with a reaction mixture comprising a first primer complementary to the target nucleic acid, a modified thermostable enzyme, such as a modified thermostable polymerase (e.g., a modified thermostable DNA polymerase or a modified thermostable RNA polymerase), and nucleotides (e.g., ribonucleotides or deoxyribonucleotides), incubating the resulting mixture at a temperature greater than about 50° C. for a period of time sufficient to activate the modified thermostable polymerase so that the polymerase produces primer extension products from the first primer and the target nucleic acid.

As such, the methods of the present invention involve the use of a reaction mixture containing a reversibly modified thermostable enzyme and subjecting the reaction mixture to a high temperature incubation prior to, or as an integral part of, the nucleic acid detection methods, such as an amplification reaction. The high temperature incubation results in release of the carboxylic acid group and activation of the thermostable enzyme.

The release of the carboxylic acid group from the modified amino groups results from both the increase in temperature and a concomitant decrease in pH. Amplification reactions typically are carried out in a Tris-HCl buffer formulated to a pH of 7.0 to about pH 9.0 at room temperature. At room temperature, the alkaline reaction buffer conditions favor the modified form of the thermostable enzyme. Although the pH of the reaction buffer is adjusted to a pH of 7.0 to 9.0 at room temperature, the pH of a Tris-HCl reaction buffer decreases with increasing temperature. The change in pH which occurs resulting from the high temperature reaction conditions depends on the buffer used. The temperature dependence of pH for various buffers used in biological reactions is reported in Good et al., 1966, Biochemistry 5(2):467-477, incorporated herein by reference. For Tris buffers, the change in pKa, i.e., the pH at the midpoint of the buffering range, is related to the temperature as follows: .ΔpKa/° C.=−0.031. For example, a Tris-HCl buffer assembled at 25° C. undergoes a drop in pKa of 2.17 when raised to 95° C. for the activating incubation.

Although primer extension reactions (e.g., amplification reactions) are typically carried out in a Tris-HCl buffer, extension reactions may be carried out in buffers which exhibit a smaller or greater change of pH with temperature. Depending on the buffer used, a more or less stable modified enzyme may be desirable. For example, using a modifying reagent which results in a less stable modified enzyme allows for recovery of sufficient enzyme activity under smaller changes of buffer pH. An empirical comparison of the relative stabilities of enzymes modified with various reagents, as provided above, guides selection of a modified enzyme suitable for use in particular buffers.

In the methods of the present invention, activation of the modified enzyme is achieved by an incubation carried out at a temperature which is equal to or higher than the primer hybridization (annealing) temperature used in the extension reaction to insure extension specificity. The length of incubation required to recover enzyme activity depends on the temperature and pH of the reaction mixture and on the stability of the modified thermostable enzyme, which depends on the modifier reagent used in the preparation of the modified enzyme. A wide range of incubation conditions are usable; optimal conditions are determined empirically for each reaction. In general, an incubation is carried out in the amplification reaction buffer at a temperature greater than about 50° C. for between about 10 seconds and about 20 minutes. Optimization of incubation conditions for the reactivation of enzymes not exemplified, or for reaction mixtures not exemplified, can be determined by routine experimentation following the guidance provided herein.

As will be readily apparent, design of the assays described herein is subject to a great deal of variation, and many formats are known in the art. The following descriptions are merely provided as guidance and one of skill in the art can readily modify the described protocols, using techniques well known in the art.

Invader Assay

In some embodiments, the reversibly modified thermostable enzyme is a reversibly modified thermostable nuclease, such as a thermostable endonuclease. In such embodiments, the reversibly modified thermostable nuclease can be used in a nucleic acid signal detection assay, such as the invader assay. The invader assay is a signal amplification method disclosed in U.S. Pat. Nos. 6,348,314; 6,090,543; 6,001,567; 5,985,557; 5,846,717; and 5,837,450, the disclosures of which are incorporated herein by reference in their entirety. It does not involve target nucleic acid sequence amplification or modification. In its linear form, two partially overlapped oligonucleotides hybridize to a target nucleic acid molecule and form a cleavable structure. Detectable signal is generated by enzymatic cleavage of the hybridized probe. The cleavage event also thermodynamically promotes removal of the cleaved probe from the target sequence. The probe undergoes a cycle of hybridization and cleavage in the presence of the target nucleic acid sequence. Signal intensity is linearly proportional to the amount of target nucleic acid sequence present in a sample. In a serial cleavage, a cleaved product from the first reaction further forms a second cleavage structure with two other oligonucleotides. Cleavage of the second cleavage structure provides further signal amplification (Hall et al, 2000, PNAS, 97(15):8272-8277). The enzyme carrying out cleavage of the hybridized probe is a thermostable flap endonuclease. Like other thermostable enzyme, flap endonuclease is active in a broad range of temperatures and is capable of cleaving many nucleic acid structures in addition to the desired cleavage target nucleic acid structures. Oligonucleotides present in a reaction system, some at high concentration, could form a variety of intra-molecular and inter-molecular structures. Most of them are only stable at low temperature. Cleavage of those structures results in either high background or low detectable signal. To reduce or even eliminate these unwanted cleavages could dramatically improve quality of the detection assay. Chemical modification, as disclosed herein, of the flap endonuclease is a good way to avoid the problems. Although it does not prevent the oligonucleotides from forming the cleavage structures, it does prevent the structures from being cleaved. At reaction temperature, the structures are unlikely stable enough to cause any trouble for the detection assay as described above.

RNA molecules are sensitive to heat, particularly in the presence of divalent metal ions. Therefore, use of a reversibly modified (e.g. reversibly inactivated) thermostable endonuclease would be ideal. However, current methods require prolonged incubation at high temperature, e.g., 95° C. in order to achieve activation. Such conditions increase the chances of the breakdown of RNA molecules, which will indirectly decrease the detection sensitivity. Accordingly, the present invention provides a chemical modification method with a large pool of modifiers. This large pool of modifiers makes it possible to choose a modifier that can form an amide bond with appropriate stability so activation can be carried out under a milder condition. This represents an important advantage of the present invention over the previous chemical modification methods.

Cycling Probe Assay (CPA)

In cycling probe assay, as disclosed in U.S. Pat. Nos. 5,403,711; 5,011,769, RNase H enzyme, preferentially a thermostable one, and a probe containing ribonucleotide(s) are used for DNA sequence detection. RNase H is an enzyme that specifically cleaves ribonucleotide molecules hybridized to deoxyribonucleotide molecules. Cleavage of the ribonucleotide molecules provides for the disassociation of the RNA molecule from the DAN molecule. Subsequently new intact RNA probes will bind to the target sequence and get cleaved. Repeating this process results in generation of detectable signal. Although the optimal temperature of activity of a thermostable RNase H is high, it usually has a significant level of activity at low temperatures. Non-specific hybridization of ribonucleotide-containing probes will trigger enzymatic cleavage of the hybridized probe by RNase H resulting in either a high background or false positive results. Reversibly modified thermostable RNase H according to the present invention will significantly improve the assay.

Polymerase Chain Reaction (PCR)

The methods of the present invention are particularly suitable for the reduction of non-specific amplification in a PCR. However, the invention is not restricted to any particular amplification system.

In a representative embodiment, a PCR amplification is carried out using a reversibly inactivated thermostable DNA polymerase. The annealing temperature used in a PCR amplification typically is about 55° C.-75° C., and the pre-reaction incubation is carried out at a temperature equal to or higher than the annealing temperature, preferably a temperature greater than about 90° C. The amplification reaction mixture preferably is incubated at about 90° C.-100° C. for up to about 12 minutes to activate the DNA polymerase prior to the temperature cycling. The period of time can be anywhere between about 5 second to about 12 minutes, including about 30 seconds to about 11 minutes, about 45 second to about 10.5 minutes, about 1 minute to about 10 minutes, about 1.5 minute to about 9.4 minutes, about 2 minutes to about 9 minutes, about 2.5 minutes to about 8.5 minutes, from about 3 minutes to about 8 minutes, from about 3.5 minutes to about 7.5 minutes, from about 4 minutes to about 7 minutes, from about 4.5 minutes to about 6.5 minutes, from about 5 minutes to about 6 minutes. Suitable pre-reaction incubation conditions for typical PCR amplifications are described in the Examples, along with the effect on amplification of varying the pre-reaction incubation conditions.

The first step in a typical PCR amplification consists of heat denaturation of the double-stranded target nucleic acid. The exact conditions required for denaturation of the sample nucleic acid depends on the length and composition of the sample nucleic acid. Typically, incubation at 90° C.-100° C. for about 10 seconds up to about 4 minutes is effective to fully denature the sample nucleic acid. The initial denaturation step can serve as the pre-reaction incubation to activate the reversibly modified thermostable DNA polymerase. However, depending on the length and temperature of the initial denaturation step, and on the modifier used to inactivate the DNA polymerase, recovery of the DNA polymerase activity may be incomplete. If maximal recovery of enzyme activity is desired, the pre-reaction incubation may be extended or, alternatively, the number of amplification cycles can be increased.

In a certain embodiments of the invention, the modified enzyme and initial denaturation conditions are chosen such that only a fraction of the recoverable enzyme activity is recovered during the initial incubation step. Subsequent cycles of a PCR, which each involve a high-temperature denaturation step, result in further recovery of the enzyme activity. Thus, activation of enzyme activity is delayed over the initial cycling of the amplification. This “time release” of DNA polymerase activity has been observed to further decrease non-specific amplification. It is known that an excess of DNA polymerase contributes to non-specific amplification. In the present methods, the amount of DNA polymerase activity present is low during the initial stages of the amplification when the number of target sequences is low, which reduces the amount of non-specific extension products formed. Maximal DNA polymerase activity is present during the later stages of the amplification when the number of target sequences is high, and which enables high amplification yields. If necessary, the number of amplification cycles can be increased to compensate for the lower amount of DNA polymerase activity present in the initial cycles. The effect on amplification of varying the amplification cycle number is shown in the Examples.

An advantage of the methods of the present invention is that the methods require no manipulation of the reaction mixture following the initial preparation of the reaction mixture. Thus, the methods are ideal for use in automated amplification systems and with in-situ amplification methods, wherein the addition of reagents after the initial denaturation step or the use of wax barriers is inconvenient or impractical.

Sample preparation methods suitable for each primer extension reaction, including amplification reaction, are described in the art (see, for example, Sambrook et al., supra, and the references describing the amplification methods cited above). Simple and rapid methods of preparing samples for the PCR amplification of target sequences are described in Higuchi, 1989, in PCR Technology (Erlich ed., Stockton Press, New York), and in PCR Protocols, Chapters 18-20 (Innis et al., ed., Academic Press, 1990), both incorporated herein by reference. One of skill in the art will be able to select and empirically optimize a suitable protocol.

Methods for the detection of amplified products have been described extensively in the literature. Standard methods include analysis by gel electrophoresis or by hybridization with oligonucleotide probes. The detection of hybrids formed between probes and amplified nucleic acid can be carried out in variety of formats, including the dot-blot assay format and the reverse dot-blot assay format. (See Saiki et al, 1986, Nature 324:163-166; Saiki et al., 1989, Proc. Natl. Acad. Sci. USA 86:6230; PCT patent Publication No. 89/11548; U.S. Pat. Nos. 5,008,182, and 5,176,775; PCR Protocols: A Guide to Methods and Applications (ed. Innis et al., Academic Press, San Diego, Calif.):337-347; each incorporated herein by reference. Reverse dot-blot methods using microwell plates are described in copending U.S. Ser. No. 141,355; U.S. Pat. No. 5,232,829; Loeffelholz et al., 1992, J. Clin. Microbiol. 30(11):2847-2851; Mulder et al., 1994, J. Clin. Microbiol. 32(2):292-300; and Jackson et al., 1991, AIDS 5:1463-1467, each incorporated herein by reference.

Ligase Chain Reaction (LCR)

Similar to PCR, LCR (Wu and Wallace, 1989, Genomics 4:560-569 and Barany, 1991, Proc. Natl. Acad. Sci. USA 88:189-193) is an exponential target amplification method involving thermocycling. Low sensitivity detection associated with LCR is at least partially attributed to residual activity of a thermostable ligase at a temperature below its reaction temperature. In LCR, non-template directed amplification is indistinguishable from template-directed amplification. Reversibly modified thermostable ligase as disclosed herein, can eliminate non-template directed ligation at low temperature.

Rolling Circle Amplification (RCA), Strand Displacement Amplification (SDA), Single Primer Isothermal Amplification (SPIA⁺), Exponential Single Primer Isothermal Amplification (X-SPIA⁺), Loop Mediated Amplification (LAMP)

Other amplification methods that can benefit from the reversibly modified thermostable enzymes of the present invention include, but are not limited to the following: Rolling circle amplification (RCA) (U.S. Pat. Nos. 5,854,033, 6,183,960, 6,210,884, 6,344,329), strand displacement amplification (SDA) (U.S. Pat. No. 5,270,184), single primer isothermal amplification (SPIA⁺) (U.S. Pat. No. 5,916,779), exponential single primer isothermal amplification (X-SPIA⁺) (U.S. Pat. No. 6,251,639), loop mediated amplification (LAMP) (U.S. Pat. No. 6,410,278). A common component for all the above isothermal amplification processes is use of a DNA polymerase with strong strand displacement activity. The most widely used DNA polymerase in these technologies is Bst DNA polymerase large fragment. Most reactions are performed at a temperature between 60˜65° C. Although hot-start is expected to be able to improve amplification specificity, sensitivity and quantification, there is no hot-start system having been reported.

In general, no hot-start technology has been developed for any isothermal detection technologies. This is partially because current hot-start technologies cannot be adopted by such detection technologies. The activation process is either not complete enough or too harsh for the processes. The present invention is of a large modifier pool. Application of the present invention can achieve hot-start of amplification and improve these assays.

Nucleic Acid Sequence Based Amplification (NASBA), Transcription Mediated Amplification (TMA), and Self-Sustained Sequence Replication (3SR)

Other methods of nucleic acid detection that can benefit from the reversibly modified thermostable enzymes of the present invention include the isothermal detection methods of, for example, Nucleic Acid Sequence Based Amplification (NASBA), Transcription Mediated Amplification (TMA), and Self-Sustained Sequence Replication (3SR). Such methods are used primarily to amplify target RNA molecules at a constant temperature. Amplification comprises: (i) RNA template directed enzymatic synthesis of complementary DNA (cDNA), (ii) RNase H degradation of RNA strand in RNA/DNA heteroduplex, (iii) synthesis of double stranded DNA, (iv) synthesis of single stranded RNA by in vitro transcription, and (v) repetition of steps (i) to (iv) in order to amplify the target nucleic acid.

Since the sensitivity of these assays and the quantification of the results are not as good as PCR, application of hot-start enzyme would greatly benefit the methods. For example, a reversibly modified thermostable enzyme that is capable of activation at an elevated temperature will effectively reduce or eliminate side-reaction. This will improve the assay sensitivity. Without a hot-start system, the amplification reaction actually starts rapidly right after all components are mixed. Different amplification onset times among samples and standards, in combination with fast amplification kinetics, makes accurate and precise quantification extremely difficult. Use a reversibly modified thermostable enzyme that is capable of activation at an elevated temperature will make all amplification events begin at the same time and therefore improving the quantification.

As a general reversible protein modification process, this invention can be applied to other processes too. For example, U.S. Pat. Nos. 6,274,981 and 6,699,981 describe a process of removal of 3′phosphate of an oligonucleotide with a phosphatase and its application in PCR. Without a reversibly modified thermostable enzyme, the dephosphorylation occurs as soon as the phosphatase is mixed with a 3′phosphorylated oligonucleotide. The removal of the phosphate could have detrimental effect. Application of the current invention to that process can effectively control such reaction and improve performance.

Accordingly, the present invention is not limited to any particular nucleic acid detection system. As other systems are developed, those systems may benefit by practice of this invent-ion. For example, a survey of amplification systems was published in Abramson and Myers, 1993, Current Opinion in Biotechnology 4:41-47, incorporated herein by reference.

Kits

The present invention also provides kits, multicontainer units comprising useful components for practicing the present method. In some embodiments, the kit comprises a reversibly modified thermostable enzyme. In certain embodiments, the thermostable enzyme is thermostable polymerase, such as a thermostable DNA polymerase or a thermostable RNA polymerase, a thermostable RNase H, a thermostable DNA nuclease, such as a thermostable DNA endonuclease, a thermostable DNA ligase, thermostable reverse transcriptase, thermostable helicase, thermostable RecA, and the like. In representative embodiments, the thermostable enzyme is a thermostable DNA polymerase. In other embodiments, the thermostable enzyme is a thermostable DNA nuclease, such as a thermostable DNA endonuclease. In some embodiments, the thermostable enzyme is derived from Thermus acquaticus, Thermus thermophilus, Thermatoga maritime, Aeropyrum pernix, Aquifex aeolicus, Archaeglobus fulgidus, Bacillus caldotenax, Carboxydothermus hydrogenformans, Methanobacterium thermoautotrophicum ΔH, Methanococcus jannaschii, Methanothermus fervidus, Pyrobaculum islandicum, Pyrococcus endeavori, Pyrococcus furiosus, Pyrococcus horihoshii, Pyrococcus profundus, Pyrococcus woesei, Pyrodictium occultum, Sulfolobus acidocaldarius, Sulfolobus solfataricus, Thermoanaerobacter thermohydrosulfuricus, Thermococcus celer, Thermococcus fumicolans, Thermococcus gorgonarius, Thermococcus kodakaraensis KOD1, Thermococcus litoralis, Thermococcus peptonophilus, Thermococcus sp. 9° N-7, Thermococcus sp. TY, Thermococcus stetteri, Thermococcus zilligii, Thermoplasma acidophilum, Thermus brokianus, Thermus caldophilus GK24, Thermus flavus, Thermus rubens, or a mutant thereof.

Furthermore, additional reagents that are required or desired in the protocol to be practiced with the kit components may be present, which additional reagents include, but are not limited to: pairs of supplementary nucleic acids, single strand binding proteins, and PCR amplification reagents (e.g., nucleotides, buffers, cations, etc.), and the like. The kit components may be present in separate containers, or one or more of the components may be present in the same container, where the containers may be storage containers and/or containers that are employed during the assay for which the kit is designed.

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

Systems

Also provided are systems that find use in practicing the subject methods, as described above. For example, in some embodiments, the kit comprises a reversibly modified thermostable enzyme. In certain embodiments, the thermostable enzyme is a thermostable polymerase, such as a thermostable DNA polymerase or a thermostable RNA polymerase, a thermostable RNase H, a thermostable DNA nuclease, such as a thermostable DNA endonuclease, a thermostable DNA ligase, thermostable reverse transcriptase, thermostable helicase, thermostable RecA, and the like. In representative embodiments, the thermostable enzyme is a thermostable DNA polymerase. In other embodiments, the thermostable enzyme is a thermostable nuclease, such as a thermostable DNA endonuclease. In other embodiments, the thermostable enzyme is derived from Thermus acquaticus, Thermus thermophilus, Thermatoga maritime, Aeropyrum pernix, Aquifex aeolicus, Archaeglobus fulgidus, Bacillus caldotenax, Carboxydothermus hydrogenformans, Methanobacterium thermoautotrophicum ΔH, Methanococcus jannaschii, Methanothermus fervidus, Pyrobaculum islandicum, Pyrococcus endeavori, Pyrococcus furiosus, Pyrococcus horihoshii, Pyrococcus profundus, Pyrococcus woesei, Pyrodictium occultum, Sulfolobus acidocaldarius, Sulfolobus solfataricus, Thermoanaerobacter thermohydrosulfuricus, Thermococcus celer, Thermococcus fumicolans, Thermococcus gorgonarius, Thermococcus kodakaraensis KOD1, Thermococcus litoralis, Thermococcus peptonophilus, Thermococcus sp. 9° N-7, Thermococcus sp. TY, Thermococcus stetteri, Thermococcus zilligii, Thermoplasma acidophilum, Thermus brokianus, Thermus caldophilus GK24, Thermus flavus, Thermus rubens, or a mutant thereof.

Furthermore, additional reagents that are required or desired in the protocol to be practiced with the system components may be present, which additional reagents include, but are not limited to: pairs of supplementary nucleic acids, single strand binding proteins, and PCR amplification reagents (e.g., nucleotides, buffers, cations, etc.), and the like.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Preparation of Flap Endonuclease-1 and Tag DNA Polymerase

Archaeoglobus Fulgidus DNA was obtained from ATCC (49558D). The gene encoding Archaeoglobus Fulgidus flap endonuclease-1 (Afu FEN-1) was cloned via PCR as described by Hosfield et al. (Hosfield, 1998, JBC 275(22):16420-16427). The cloned sequence was verified by direct sequencing. The Afu FEN-1 gene was cloned into pET-28 (Novagen). Afu FEN-1 protein overexpression and purification were done according to Hosfield et al. with minor modification.

Thermus Aquaticus strain YT-1 was obtained from ATCC (25104). Thermus Aquaticus (Taq) DNA polymerase gene was cloned via PCR with sequence from GeneBank (Accession No. J04639). Plasmid pET-28 was used to construct expression vector. Purification of Taq DNA polymerase was carried out with a procedure described by Lawyer et al. (Lawyer et al., 1989, JBC 264(11):6427-37; Lawyer et al. 1989, PCR Meth. Appl. 2(4):275-87).

### Example 2

### Modification of Afu FEN-1 With Citraconic Acid

Modification OF Afu FEN-1 with citraconic acid was performed in a buffer containing 20 mM MOPS, pH8.0 and 100 mM KCl. Concentration of Afu FEN-1 was adjusted to 1 mg/ml.

Citraconic acid (Aldrich) and N,N′-dicyclohexyl carbodiimide (DCC) (Aldrich) were dissolved in N,N′-dimethyl-formamide (DMF) (Fisher, sequencing grade) at 1M. One hundred microliters of 1M citraconic acid and 200 μl 1 M DCC were mixed in a 1.5 ml Eppendorf tube. The mixture was incubated at room temperature for 1 hour. The mixture was then centrifuged at 12,000 rpm for 20 minutes at room temperature. The pellet was discarded and the supernatant was kept to modify Afu FEN-1.

One volume of activated citraconic acid was mixed with 99 volume of Afu FEN-1. The mixture was then incubated at room temperature for 1 hour to result in chemical inactivation of Afu FEN-1.

### Example 3

### Activity Assay of Modified Afu FEN-1

Modified Afu FEN-1 was tested for its flap endonuclease activity. A control reaction mixture lacking enzyme contained 30 mM Tris HCl, pH8.0, 3 mM Mg²⁺, 400 nM 5-ROX (Sigma), 0.01%Tween-20, 100 nM each of the following nucleic acids 18SI, 18SP and 18ST (see Table 1 for sequence information). Both 18SI and 18SP consists of complementary sequence to 18ST. 18SI is located upstream of 18SP and overlaps with 18SP by 1 nucleotide. Fluorescence of 6Fam of 18SP is quenched when 18SP is intact. In the presence of Afu FEN-1, 18SP in a complex containing 18SI, 18SP and 18ST is cleaved by Afu FEN-1. Such cleavage results in increase in 6FAM fluorescence. Ten nanograms of chemically modified Afu FEN-1 were added to a 25 μl reaction. The same amount of unmodified enzyme was used as a control.

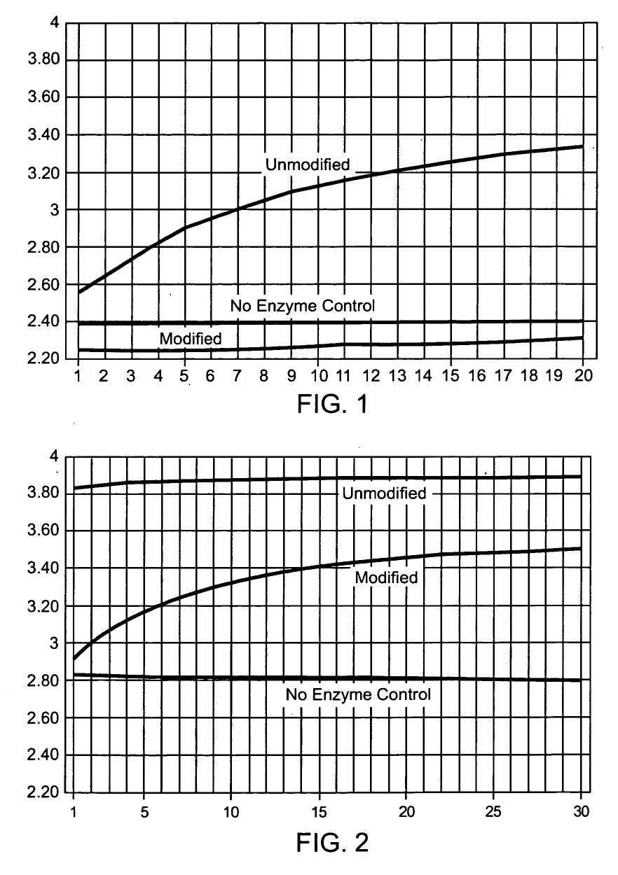

Activity assay was conducted on ABI Prism 7000 to monitor change of fluorescence intensity at real-time. Incubation condition was 20 cycles of the following: 59° C., 1 second, →60° C., 29 seconds)×20 cycles. The intended incubation condition was 60° C. for 10 minutes to collect data every 30 seconds. However, the manufacture's software does not allow this kind of operation. As shown in FIG. 1, the modified Afu FEN-1 did not display observable flap endonuclease activity.

Following the incubation, reaction mixture was further incubated at 95° C. for 10 minutes and then 30 cycles of the following: 59° C., 1 second, →60° C., 59 seconds. This is to heat activate the modified Afu FEN-1 and test its activity afterward. FIG. 2 shows that incubation at 95° C. partially restores flap endonuclease activity of the chemically modified Afu FEN-1.

TABLE 1

| | | |
|---|---|---|
| 18SI | 5′ - GGA ATG AGT CCA CTT TAA | (SEQ ID NO:01) |
| | ATC CTT TAA C - 3′ | |
| 18SP | 5′ - 6FAM CGA GGA TCC ATT GGA | (SEQ ID NO:02) |
| | GGG CAA G BHQ1 | |
| 18ST | 5′ - CTT GCC CTC CAA TGG ATC | (SEQ ID NO:03) |
| | CTC GTT AAA GGA TTT AAA GTG | |
| | GAG TCA TTC CAA TTA CAG GGC | |
| | CTC G - 3′ | |

### Example 4

### Modification of Afu FEN-1 With cis-Aconitic Acid

Modification was performed in a buffer containing 20 mM MOPS, pH8.0 and 100 mM KCl. Concentration of Afu FEN-1 was adjust to 1 mg/ml.

Cis-Aconitic acid (Aldrich) and DCC (Aldrich) were dissolved in N,N′-dimethyl-formamide (DMF) (Fisher, sequencing grade) at 1 M. 100 μl of 1M cis-aconitic acid, 100 μl 1M DCC and 100 μl DMF were mixed in a 1.5 ml Eppendorf tube. The mixture was incubated at room temperature for 1 hour. The mixture was then centrifuged at 12,000 rpm for 20 minutes at room temperature. The pellet was discarded and the supernatant was kept to modify Afu FEN-1.

One volume of activated citraconic acid was mixed with 99 volume of Afu FEN-1. Incubation at room temperature for 1 hour resulted in chemical inactivation of Afu FEN-1.

### Example 5

### Comparison of Citraconic Acid and cis-Aconitic Acid Modified Afu FEN-1

Different applications of the chemically modified enzymes require different stability of the amide bond. As such, an appropriate carboxylic acid should be chosen for the specific application. In addition, stability of the amide bond is of interest for determining storage condition of the modified protein.

Activated carboxylic acids can form amide bonds with amine group. For a particular amine group, the structure of the carboxylic acid affects the stability of the amide bond. The effect of the carboxylic acid structure on amide bond stability is reasonably predictable. For example, cis-aconitic acid contains three carboxyl groups. Each of the carboxylic groups can react with DCC to form the stable ester intermediate. However, reactivity of the three carboxyl groups is not equal. 3-carboxyl group of cis-aconitic acid is predicted the most reactive group with zero-length cross-linker due to stereo effect. When the molar ratio of cis-aconitic acid to DCC in a reaction mixture is about 1, there are three carboxyl groups for every DCC molecule. The active ester that is formed between DCC and the 3-carboxyl group is to expected to be at a higher concentration than the active ester that is formed from either one of the other two carboxylic acid groups.

Although the structure of the activated carboxylic acid can be determined by various analytical methods. According to Palacian (Palacian et al., 1990, MCB, 97:101-111), the amide bond formed with the 3-carboxyl group is more stable and more difficult to be broken down than the amide bonds formed with the other two carboxyl groups. The deacylation reaction should be even more difficult than that with citraconic acid. Therefore relative easiness of activation can reveal the composition of the activated carboxylic acid.

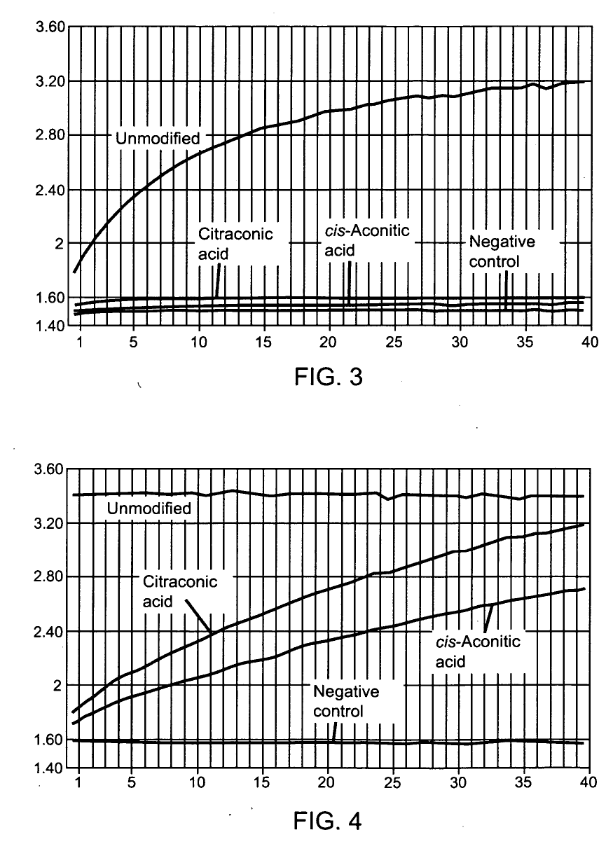

Modified Afu FEN-1 with carboxylic acid or cis-aconitic acid was prepared as in Example 2 and 4. Flap endonuclease-1 assay and activation conditions are described in Example 3. FIG. 3 shows that both cis-aconitic acid modified enzyme as well as citraconic acid modified enzyme did not have any significant flap endonuclease activity. However, after activation, as demonstrated in FIG. 4, both modified enzymes can be activated by incubation at 95° C. for 10 minutes. As shown in FIG. 4 flap endonuclease activity of the citraconic acid modified Afu FEN-1 was restored 60˜70% more than the cis-aconitic acid modified Afu FEN-1.

### Example 6

### Modification of FEN-1 with NHS Ester of Citraconic Acid

DCC, citraconic acid and NHS (Aldrich) were all dissolved in DMF at 1M. 200 μl of DCC, 200 μl NHS and 100 μl of citraconic acid were mixed in a 1.5 ml tube. The mixture was then incubated at room temperature for 1 hour. The mixture was then centrifuged at 12,000 rpm for 20 minutes at room temperature. The pellet was discarded and the supernatant was kept to modify Afu FEN-1.

Afu FEN-1 to be modified was kept in a buffer containing 20 mM MOPS, pH8.0 and 100 mM KCl. Concentration of Afu FEN-1 was adjusted to 1 mg/ml. One volume of activated citraconic acid was mixed with 99 volume of Afu FEN-1. The mixture was then incubated at room temperature for 1 hour in order to result in inactivation of Afu FEN-1.

### Example 7

### Modification of FEN-1 with Sulfo-NHS Ester of Citraconic Acid

Sulfo-NHS ester is commonly used in acylation reactions. The sulfo-NHS ester has the same specificity and reactivity as NHS ester. The difference between sulfo-NHS ester and NHS ester is water solubility and stability of the compounds in an aqueous solution. In particular, sulfo-NHS ester is more hydrophilic than NHS ester. Therefore, hydrolysis of sulfo-NHS ester in aqueous solution is slower than NHS ester. As such, it is advantageous to use sulfo-NHS ester to mediate acylation reaction.

DCC, citraconic acid and sulfo-NHS (Pierce) were all dissolved in DMF at 1M. 200 μl of DCC, 200 μl of sulfo-NHS and 100 μl of citraconic acid were mixed in a 1.5 ml tube. The mixture was incubated at room temperature for 1 hour. The mixture was then centrifuged at 12,000 rpm for 20 minutes at room temperature. The pellet was discarded and the supernatant was kept to modify Afu FEN-1.

Afu FEN-1 to be modified was kept in a buffer containing 20 mM MOPS, pH8.0 and 100 mM KCl. Concentration of Afu FEN-1 was adjust to 1 mg/ml. One volume of activated citraconic acid was mixed with 99 volume of Afu FEN-1. The mixture was then incubated at room temperature for 1 hour in order to result in inactivation of Afu FEN-1.

### Example 8

### Modification of Tag DNA Polymerase with Citraconic Acid

DCC, citraconic acid and NHS (Aldrich) were all dissolved in DMF at 1M. 200 μl of DCC, 200 μl of NHS and 100 μl of citraconic acid were mixed in a 1.5 ml tube. The mixture was then incubated at room temperature for 1 hour. The mixture was then centrifuged at 12,000 rpm for 20 minutes at room temperature. The pellet was discarded and the supernatant was kept to modify Afu FEN-1.

Purified Taq DNA polymerase is then adjusted to 1 mg/ ml in 20 mM MOPS, pH8.0 and 100 mM KCl. One volume of activated citraconic acid was mixed with 99 volume of Taq DNA polymerase. The mixture was then incubated at room temperature for 1 hour in order to result in inactivation of Taq DNA polymerase.

### Example 9

### pH Dependence of Activation of Modified Enzyme

It has been reported that both higher temperature and lower pH facilitate deacylation reaction (Nieto et al., 1983, Biochem. Biophys. Acta., 749:204-210). Both factors are present in a hot start PCR system. Tris buffer, the most commonly used buffer in PCR, becomes significantly more acidic when the temperature rises. It has been determined that pH lowers 0.031 per degree (° C.) increase. For example, if a Tris solution is pH 8.0 at 22° C., the pH of the solution drops down to 5.74 when the temperature reaches 95° C.

Modified Taq DNA polymerase was tested for its pH dependence of activation. A 25 μl PCR reaction mixture contained 25 mM Tris, pH either 8.0 or 8.7, 30 mM KCl, 3.0 mM Mg²⁺, 0.2 mM each dATP, dCTP, dGTP and TTP, 400 nM 5-ROX, 1×Sybr Green, 0.30 ng human genomic DNA from K562 cells (Promega), 200 nM each primer (see Table 2 for sequence information), and 10 ng unmodified or modified Taq DNA polymerase. Target amplified was 18S ribosomal RNA gene. All reactions were carried out on one plate. Thermocycling conditions included 95° C. for 10 minutes followed by 40 cycles of 95° C., 15 seconds→60° C., 30 seconds. Amplification was performed on an ABI Prism 7000.

TABLE 2

| | | |
|---|---|---|
| 18SF | 5′ - CGA GGC CCT GTA ATT GGA | (SEQ ID NO:04) |
| | A - 3′ | |
| 18SR | 5′ - CGG CTG CTG GCA CCA GA - 3′ | (SEQ ID NO:05) |

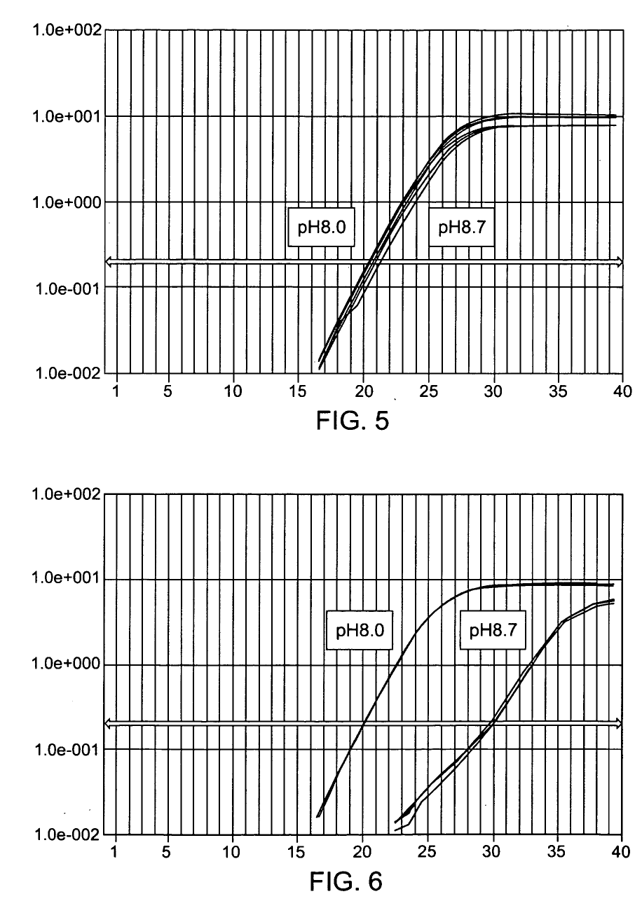

FIG. 5 shows amplification with unmodified enzyme. Neither cycle threshold (Ct) nor ΔRn were significantly affected by pH. FIG. 6 shows amplification with modified Taq DNA polymerase. In contrast to unmodified Taq DNA polymerase, amplification with modified Taq DNA polymerase was greatly impacted by pH. For example, Ct with the pH 8.7 system shifted nearly 10 cycles higher than with a pH 8.0 system. The results show the importance of pH for activation of modified enzyme.

### Example 10

### PCR Amplification with Modified Tag DNA Polymerase in a Non-Tris Buffer System

Although chemically modified DNA polymerase provides the most stringent hot start capability, the use of chemically modified DNA polymerase in PCR has been limited to amplification of small fragments. Another factor in achieving optimal PCR amplification is pH. Buffer pH for unmodified thermostable DNA polymerases is usually between pH 8.3˜9.0 depending on the origin of the enzyme and the formulation by each commercial vendor. No single commercial buffer for PCR has a pH lower than pH 8.0. In addition, a buffer pH 8.0 is actually sub-optimal for polymerase activity. Sub-optimal pH is an important factor in why, for example, AmpliTaq Gold cannot not amplify large nucleic acids.

For high fidelity PCR amplification, thermostable DNA polymerase with proofreading activity, e.g. Pfu DNA polymerase (Stratagene), Vent & Deep Vent DNA polymerase (New England Biolabs), can be used. In general this kind of enzyme prefers a higher pH buffer system, such as pH 8.8, to achieve high fidelity amplification of large nucleic acids.

In particular, the effect of pH in the efficiency of PCR is most significantly seen at the DNA synthesis step. For large fragment amplification, the preferred temperature for primer extension is 72° C. For small fragment amplification, 2-step PCR is the most common, wherein primer annealing and primer extension are usually done at 60° C.

Moreover, the effect of temperature on the pH of different buffer systems varies. For example, when temperature goes up one degree of centigrade, pH of Tris and MOPS drops 0.031 and 0.009 respectively. Table 3 shows pH of Tris and MOPS buffer at different temperature. In Table 3, pH at 22° C. can be measured with a pH meter. pH at other temperatures are calculated based on pKa change with each buffer.

TABLE 3

PH

| Buffer | 22° C. | 60° C. | 72° C. | 95° C. |
|---|---|---|---|---|
| Tris | 8.00 | 6.82 | 6.45 | 5.74 |
| Tris | 8.80 | 7.62 | 7.25 | 6.54 |
| MOPS | 7.25 | 6.91 | 6.80 | 6.59 |
| MOPS | 7.50 | 7.16 | 7.05 | 6.84 |
| MOPS | 7.75 | 7.41 | 7.30 | 7.09 |

According to Table 3, if a MOPS buffer has pH of about 7.25 to 7.50 at 22° C., the pH of the buffer at 60° C. would be 6.91 to 7.16. Such a pH should be good for amplification of small fragments. If a MOPS buffer has a pH of about 7.50 to 7.75, for the buffer is suitable for use in amplifying large nucleic acid fragments. To determine whether the modified enzymes of the present invention can be used in large nucleic acid fragment amplification or high fidelity nucleic acid amplification, different MOPS buffer system were tested for their suitability.

An obstacle to applying the subject enzymes to different applications is if the pH of different reaction systems could allow for effective activation of the modified enzyme. To address this issue, a set of experiments was designed as follows.

A 25 μl PCR reaction mixture contained 25 mM Tris, pH 8.0 or 25 mM MOPS pH 7.25, 7.50, and 7.75. The rest components are 30 mM KCl, 3.0 mM Mg²⁺, 0.2 mM each dATP, dCTP, dGTP and TTP, 400 nM 5-ROX, 1×Sybr Green, 0.30 ng human genomic DNA from K562 cells (Promega), 200 nM each primer (see Table 2 for sequence information), and 10 ng unmodified or modified Taq DNA polymerase. The target that was amplified was the 18S ribosomal RNA gene. All reactions were carried out on one plate. Thermocycling condition were 95° C. for 10 minutes and 40 cycles of the following: 95° C., 15 seconds→60° C., 30 seconds. Amplification was performed on an ABI Prizm 7000. The results are provided in Table 4.

TABLE 4

| | |
|---|---|
| Cycle Threshold (Ct) | ΔRn |

| | | Stdev, | | Stdev, |
|---|---|---|---|---|
| Buffer/pH | Average | n = 3 | Average | n = 3 |
| Tris/8.00 | 22.63 | 0.09 | 16.4 | 0.5 |
| MOPS/7.25 | 21.92 | 0.06 | 17.7 | 1.1 |
| MOPS/7.50 | 21.94 | 0.03 | 20.4 | 0.4 |
| MOPS/7.75 | 22.52 | 0.05 | 17.6 | 1.2 |

In contrast to modified Taq DNA polymerase in pH 8.70 Tris buffer, in which the enzyme cannot be activated well (Example 9), the same modified Taq DNA polymerase in MOPS buffers with pH from 7.25 to 7.75 was activated and functioned well. The results show that the reversibly modified thermostable enzymes of the subject invention can be used in varying amplification processes.

### Example 11

### Preparation and Modification of A Truncated Taq DNA polymerase

Taq DNA polymerase has two domains. The first domain is a DNA polymerase domain and the second domain is a 5′→3′ nuclease domain. Deletion of N-terminal nuclease domain produces a truncated Taq DNA polymerase with higher replication fidelity and thermostability (Barnes, 1992, Gene 112:29-35; Lawyer et al., 1993, PCR Methods App. 2(4):275-287). The truncated Taq DNA polymerase has successfully been used in amplification of large nucleic acid fragments.

The gene encoding the truncated Taq DNA polymerase (Barnes, 1992) was subcloned into pET-28 expression vector. The pET-28 expression vector containing the deletion mutant was then transformed into a BL21 (DE3) cell line in order to express the truncated Taq DNA polymerase. The purification protocol described by Lawyer was adopted for purification of overexpressed truncated Taq DNA polymerase (Lawyer, 1993).

Modification of the recombinant truncated Taq DNA polymerase was performed as described in Example 2.

### Example 12

### Quantitative PCR Using A DNA Polymerase and Afu FEN-1 Endonuclease

Quantitative PCR using a DNA polymerase lacking a 5′ nuclease activity and a flap endonuclease-1 and described in U.S. Pat. Nos. 6,528,254, and 6,548,250, the disclosures of which are incorporated herein by reference in their entirety. The endonuclease FEN-1 is capable of cleaving many secondary structures, such as cleaving primers and/or probes that form intra-molecular or inter-molecular secondary structures. If such cleavage occurs, it could negatively impact amplification and/or signal detection. Such intra- or inter-molecular structures are more stable at lower temperatures than at higher temperatures. Therefore, cleavage by the endonuclease is more likely to occur at low temperature. As such, a reversibly chemically modified FEN-1 that becomes active at elevated temperatures is very helpful in reduce or even prevent such cleavage events at the lower temperatures. Consequently amplification and signal detection can be improved using such a reversibly chemically modified endonuclease.

A 25 μl PCR reaction mixture contained 15 mM Tris, pH 8.0, 4.0 mM Mg²⁺, 0.2 mM each DATP, dCTP, dGTP and TTP, 400 nM 5-ROX, 1×Sybr Green, 1.5 ng human genomic DNA (ABI), 400 nM of each primer, and 100 nM probe (see Table 5 for sequence information), and 10 ng modified truncated Taq DNA polymerase (Example 11), and 6 ng or 10 ng either unmodified or modified Afu FEN-1. Target amplified was a fragment of a gene on chromosome 10. Thermocycling conditions included 25° C., 15 minutes→95° C., 10 minutes, and 45 cycles of the following: 95° C.,15 seconds→60° C., 1 minute. Amplification was performed on an ABI Prism 7000.

TABLE 5

| | | |
|---|---|---|
| Forward | 5′ - TGC TGA ATT TCC ATC TGT GAG TTC - 3′ | (SEQ ID NO:06) |
| Reverse | 5′ - GCA GGA TTC AGT GCC AGA AAG - 3′ | (SEQ ID NO:07) |
| Probe | 5′ - FAM-TAC CAC GCT TTT TC-DQ-MGB - 3′ | (SEQ ID NO:08) |

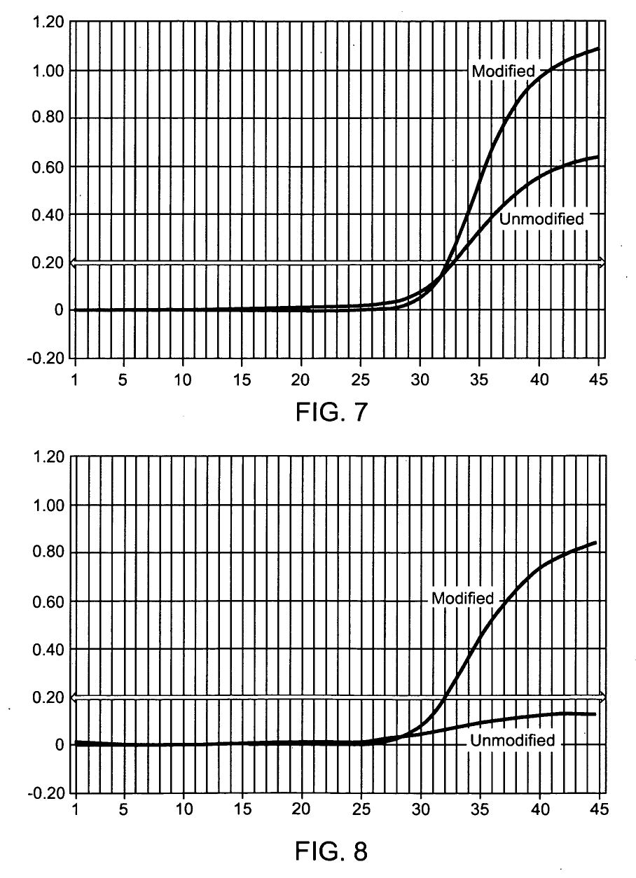

While PCR with 6 ng of unmodified Afu FEN-1 was successful in detecting the target nucleic acid, the reaction produced a significantly weaker signal than the reaction containing the reversibly modified endonuclease (FIG. 7). The difference between modified and unmodified was even more dramatic when the concentration of Afu FEN-1 that was used in the reaction was increase to 10 ng. The results show that unlike 10 ng of unmodified Afu FEN-1 that totally failed to detect the target nucleic acid, detection with 10 ng modified Afu FEN-1 was successful (FIG. 8).

### Example 13

### Comparison of Carboxylic Acid Modified DNA Polymerase to Dicarboxylic Acid Anhydride Modified DNA Polymerase

The following study compared the efficacy (e.g., speed of the reaction and sensitivity of the reaction) of a reversible thermostable DNA polymerase of the subject invention and a polymerase modified using a dicarboxylic acid anhydride as described in U.S. Pat. No. 5,677,152.

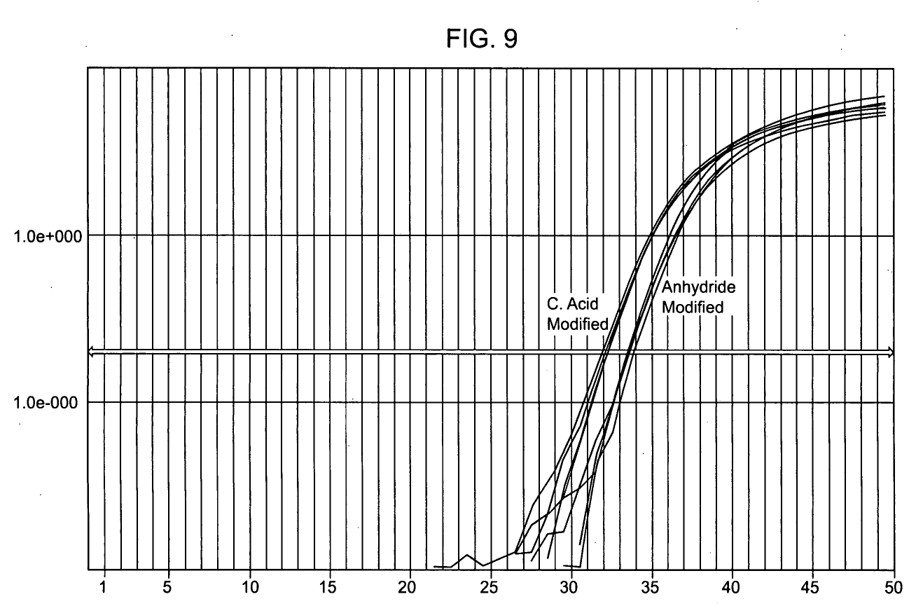

A 25 μl PCR reaction mixture contained Tris buffer, pH 8.0, 4.0 mM Mg²⁺, 0.2 mM each DATP, dCTP, dGTP and TTP, 400 nM 5-ROX, 300 pg human genomic DNA (ABI), 200, 400, or 800 nM of each primer, and 200 nM probe (see Table 6 for sequence information and amount of each primer added), and 10 ng of modified Taq DNA polymerase (Example 8), Univesal TaqMan PCR Master Mix (Part Number 4304437) was purchased from Applied Biosystem (ABI). The master mix contains AmpliTaq Gold, a Taq DNA polymerase modified with dicarboxylic acid anhydride. The targets that were amplified are listed in Table 6. The standard ABI thermocycling protocol was 95° C., 10 minutes then 50 cycles of the following: 95° C., 15 seconds→60° C., 1 minute. The Fast thermocycling protocol was 95° C., 5 minutes, then 50 cycles of the following: 95° C., 5 seconds→+60° C., 30 seconds. Amplification was performed on an ABI Prism 7000. The results are provided in Tables 7 to 9 and FIGS. 9-11.

TABLE 6

| | | |
|---|---|---|
| Target | Sequence | NM |

| | | | |
|---|---|---|---|
| Target 1 | | | |
| Forward | 3′ - GGCAAAGAACAGAAGTAAAATCCAGAA - 5′ | (SEQ ID NO:09) | 400 |
| Reverse | 3′ - CAGTTTCACAGTGAAAGTTGGCAA - 5′ | (SEQ ID NO:10) | 400 |
| Probe | 3′ - 6FAM-TGCCTCAAGCAGC-MGB-DQ - 5′ | (SEQ ID NO:11) | 200 |
| Target 2 | | | |
| Forward | 3′ - TGGGCCTGACCACTCCTTT - 5′ | (SEQ ID NO:12) | 800 |
| Reverse | 3′ - TGCGATCCCGCTTGTGAT - 5′ | (SEQ ID NO:13) | 800 |
| Probe | 3′ - 6FAM-TGCCCAGCCCCAG-MGB-DQ - 5′ | (SEQ ID NO:14) | 200 |
| Target 3 | | | |
| Forward | 3′ - CAGGTGGAGACCCTGAGAA - 5′ | (SEQ ID NO:15) | 400 |
| Reverse | 3′ - ACACCTTTGGTCACTCCAAAT - 5′ | (SEQ ID NO:16) | 400 |
| Probe | 3′ - 6FAM-TCCCAGAGCTCCCAGGGTCC-BHQ1 - 5′ | (SEQ ID NO:17) | 200 |
| Target 4 | | | |
| Forward | 3′ - GCGGAGGGAAGCTCATCAG - 5′ | (SEQ ID NO:18) | 400 |
| Reverse | 3′ - CCCTAGTCTCAGACCTTCCCAA - 5′ | (SEQ ID NO:19) | 400 |
| Probe | 3′ - 6FAM-CCACGAGCTGAGTGCGTCCTGTCA- | (SEQ ID NO:20) | 200 |
| | BHQ1- 5′ | | |
| Target 5 | | | |
| Forward | 3′ - CATTCCTCTGCAGCACTTCACT - 5′ | (SEQ ID NO:21) | 400 |
| Reverse | 3′ - CGGTTCAGTCCACATAATGCAT - 5′ | (SEQ ID NO:22) | 400 |
| Probe | 3′ - 6FAM-CAAATGAGCATTAGC-MGB-DQ - 5′ | (SEQ ID NO:23) | 200 |
| Target 6 | | | |
| Forward | 3′ - GAAACGCATCTCACTGTGATTCTATT - 5′ | (SEQ ID NO:24) | 400 |
| Reverse | 3′ - CACCATACTTCATGGCAAGGACT - 5′ | (SEQ ID NO:25) | 400 |
| Probe 1 | 3′ - 6FAM-CACCATTAGATCCTG-MGB-DQ - 5′ | (SEQ ID NO:26) | 200 |
| (Allele 1) | | | |
| Probe 2 | 3′ - VIC-CACCATTAGGTCCTG-MGB-DQ - 5′ | (SEQ ID NO:27) | 200 |
| (Allele 2) | | | |
| Target 7 | | | |
| Forward | 3′ - GAGGTTTCACTGGCTTGTGCT - 5′ | (SEQ ID NO:28) | 400 |
| Reverse | 3′ - CATGAGACATTTATCTAATGATTTTTTCTTA | (SEQ ID NO:29) | 400 |
| | TT- 5′ | | |
| Probe 1 | 3′ - 6FAM-CCATGCGTTAGCC-MGB-DQ - 5′ | (SEQ ID NO:30) | 200 |
| (Allele 1) | | | |
| Probe 2 | 3′ - VIC-CCATGGGYTTAGCCAA-MGB-DQ - 5′ | (SEQ ID NO:31) | 200 |
| (Allele 2) | | | |
| Target 8 | | | |
| Forward | 3′ - TGCTGAATTTCCATCTCTGAGTTC - 5′ | (SEQ ID NO:32) | 400 |
| Reverse | 3′ - GCAGGATTCAGTGCCAGAAAG - 5′ | (SEQ ID NO:33) | 400 |
| Probe 1 | 3′ - 6FAM-TACCACGCTTTTTC-MGB-DQ - 5′ | (SEQ ID NO:34) | 200 |
| (Allele 1) | | | |
| Probe 2 | 3′ - VIC-TGTACCACTCTTTTTC-MGB-DQ - 5′ | (SEQ ID NO:35) | 200 |
| (Allele 2) | | | |

TABLE 7
Comparison of Fast Thermocycling Protocol
vs. Standard Thermocycling Protocol for the
Carboxylic Acid Modified DNA Polymerase

| | | Fast | | Standard | |
|---|---|---|---|---|---|
| | | Ct, Ave | Stdev, n = 4 | Ct, Ave | Stdev, n = 4 |
| Target 1 | | 32.03 | 0.06 | 31.97 | 0.16 |
| Target 2 | | 33.43 | 0.42 | 33.34 | 0.21 |
| Target 3 | | 32.60 | 0.15 | 32.52 | 0.35 |
| Target 4 | | 33.36 | 0.25 | 32.64 | 0.11 |
| Target 5 | | 31.82 | 0.31 | 31.86 | 0.18 |
| Target 6 | Allele 1 | 32.76 | 0.52 | 32.44 | 0.17 |
| | Allele 2 | 34.05 | 0.26 | 33.24 | 0.22 |
| Target 7 | Allele 1 | 33.45 | 0.19 | 32.90 | 0.21 |
| | Allele 2 | 33.27 | 0.16 | 32.73 | 0.20 |
| Target 8 | Allele 1 | 33.47 | 0.46 | 33.17 | 0.47 |
| | Allele 2 | 36.00 | 0.69 | 34.46 | 0.50 |

TABLE 8
Comparison of the Carboxylic Acid Modified DNA
Polymerase and Anhydride Modified DNA Polymerase
Using the Fast Thermocycling Protocol

| | | Carboxylic Acid | | | |
|---|---|---|---|---|---|
| | | Modified | | Anhydride Modified | |
| | | Ct, Ave | Stdev, n = 4 | Ct, Ave | Stdev, n = 4 |
| Target 1 | | 32.03 | 0.06 | 34.56 | 0.61 |
| Target 2 | | 33.43 | 0.42 | 35.52 | 0.13 |
| Target 3 | | 32.60 | 0.15 | 34.10 | 0.14 |
| Target 4 | | 31.82 | 0.31 | 34.27 | 0.42 |
| Target 5 | | 33.36 | 0.25 | 41.09 | 0.15 |
| Target 6 | Allele 1 | 32.76 | 0.52 | 38.17 | 0.13 |
| | Allele 2 | 34.05 | 0.26 | 40.44 | 0.28 |
| Target 7 | Allele 1 | 33.45 | 0.19 | 41.04 | 0.32 |
| | Allele 2 | 33.27 | 0.16 | 40.16 | 0.68 |
| Target 8 | Allele 1 | 33.47 | 0.46 | 48.04 | N/A* |
| | Allele 2 | 36.00 | 0.69 | N/A | N/A |

*2 out of 4 were not amplified.

TABLE 9
Comparison of the Carboxylic Acid Modified DNA Polymerase
(Fast Thermocycling Protocol) and Anhydride Modified
DNA Polymerase (Standard Thermocycling Protocol)

| | | Carboxylic Acid | | | |
|---|---|---|---|---|---|
| | | Modified | | Anhydride Modified | |
| | | (Fast Protocol) | | (Standard Protocol) | |
| | | Ct, Ave | Stdev, n = 4 | Ct, Ave | Stdev, n = 4 |
| Target 1 | | 32.03 | 0.06 | 32.78 | 0.34 |
| Target 2 | | 33.43 | 0.42 | 34.16 | 0.17 |
| Target 3 | | 32.60 | 0.15 | 33.18 | 0.12 |
| Target 4 | | 31.82 | 0.31 | 32.63 | 0.25 |
| Target 5 | | 33.36 | 0.25 | 36.15 | 0.13 |
| Target 6 | Allele 1 | 32.76 | 0.52 | 33.22 | 0.07 |
| | Allele 2 | 34.05 | 0.26 | 35.58 | 0.62 |
| Target 7 | Allele 1 | 33.45 | 0.19 | 34.86 | 0.29 |
| | Allele 2 | 33.27 | 0.16 | 34.14 | 0.17 |
| Target 8 | Allele 1 | 33.47 | 0.46 | 35.16 | 0.43 |
| | Allele 2 | 36.00 | 0.69 | 35.97 | 0.38 |

The results show that the carboxylic acid modified thermostable DNA polymerase was faster and more sensitive than the anhydride modified thermostable DNA polymerase. For example, Table 8 shows that while suing the standard thermocycling protocol, the Ct value for the carboxylic acid modified thermostable DNA polymerase was lower than the Ct value for the anhydride modified thermostable DNA polymerase. In most cases, the Ct value was from about 2 to about 14 integers lower in the carboxylic acid modified thermostable DNA polymerase mediated reaction than the anhydride modified thermostable DNA polymerase mediated reactions. Moreover, Table 9 shows that in order to achieve a comparable Ct value for the anhydride modified thermostable DNA polymerase mediated reactions, the reactions would have to performed using the standard thermocycling protocol, while the carboxylic acid modified thermostable DNA polymerase mediated reactions could be performed using the fast thermocycling protocol.

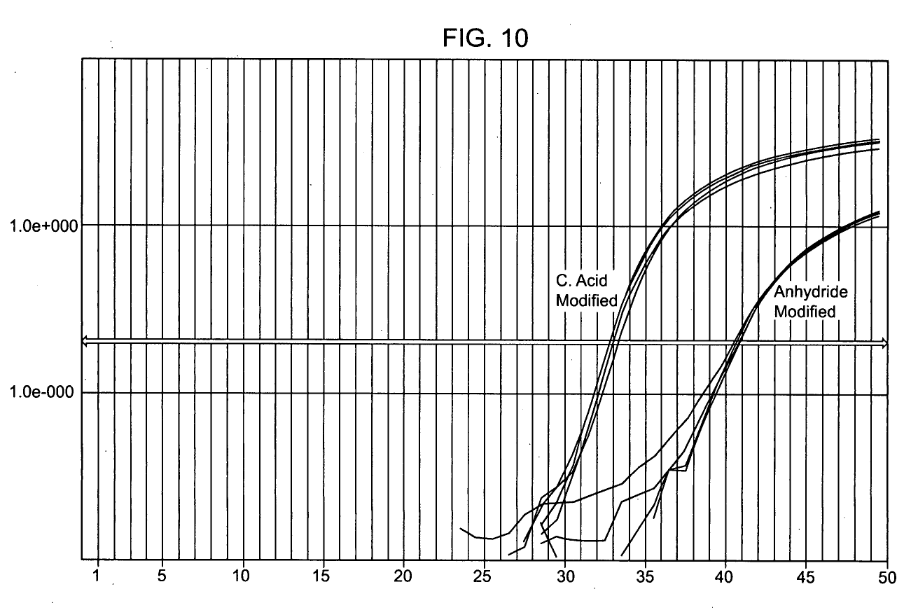

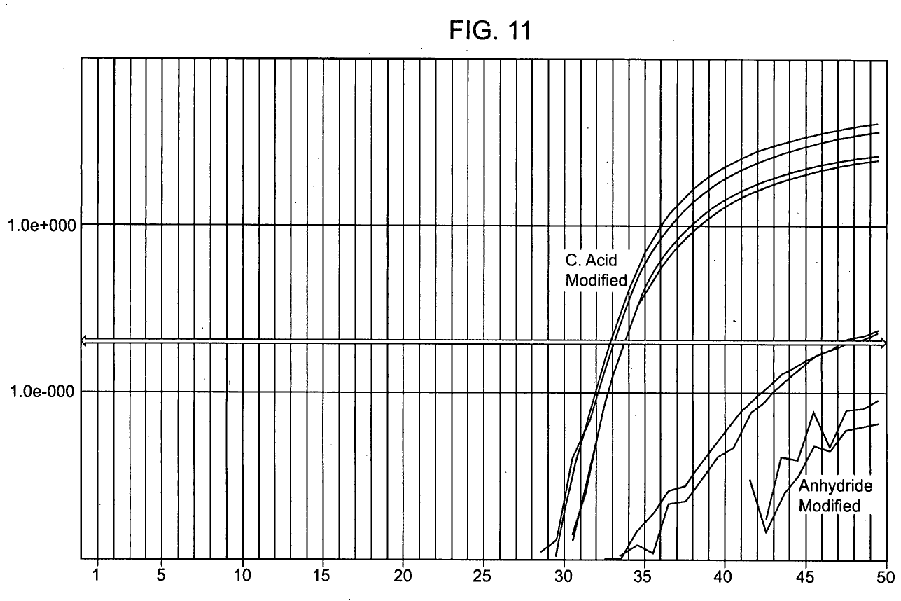

Under the standard protocol, the anhydride modified enzyme (purchased from ABI) performed nearly equally well as the carboxylic acid modified Taq DNA polymerase (Table 9). However, dramatic difference between the two systems was seen with the Fact thermocycling conditions (Table 8). FIGS. 9-11 show three representative results. FIG. 9 shows results of amplification of Target 3 under fast thermocycling conditions. Among the 8 targets compared, ABI's PCR mix works the best with Target 3. The Ct with ABI's master mix still trailed by 1.5 cycles. Greater Ct difference, 7.37, was observed with Target 5 (FIG. 10). Under the Fast Thermocycling condition, ABI's reagent essentially failed to detect the target. FIG. 11 shows results of amplification of allele 1 of Target 8.

Excluding heating and cooling time, which varies from machine to machine, the fast condition shortens reaction time by 36.3 minutes or 53%. To get fast results is very desirable in many situations, such as clinical use, detection of hazardous microbes and viruses in a suspected sample. Even in basic research use, it allows higher throughput test. Accordingly, the results show that the carboxylic acid modified thermostable enzyme worked better under fast conditions than the anhydride modified enzyme.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

That which is claimed is:

1. A thermostable enzyme composition, wherein said thermostable enzyme composition comprises a thermostable enzyme that has been covalently modified which results in essentially complete inactivation of enzyme activity,
wherein incubation of said modified thermostable enzyme composition in an aqueous buffer, formulated to about pH 7 to about pH 9 at 25° C., at a temperature greater that about 50° C. results in at least a two-fold increase in activity of the composition in less than about 20 minutes.

2. The thermostable enzyme composition according to claim 1, wherein said thermostable enzyme is a thermostable DNA polymerase, a thermostable RNA polymerase, a thermostable RNase H, a thermostable nuclease, or a thermostable DNA ligase, a thermostable reverse transcriptase, a thermostable RecA, a thermostable helicase.

3. The thermostable enzyme composition according to claim 1, wherein said thermostable enzyme is a thermostable polymerase.

4. The thermostable enzyme composition according to claim 3, wherein said thermostable polymerase is a thermostable DNA polymerase.

5. The thermostable enzyme composition according to claim 1, wherein said thermostable polymerase is a thermostable RNA polymerase.

6. The thermostable enzyme composition according to claim 1, wherein said thermostable enzyme is a thermostable nuclease.

7. The thermostable enzyme composition according to claim 1, wherein said thermostable enzyme is derived from Thermus acquaticus, Thermus thermophilus, Thermatoga maritime, Aeropyrum pernix, Aquifex aeolicus, Archaeglobus fulgidus, Bacillus caldotenax, Carboxydothermus hydrogenformans, Methanobacterium thermoautotrophicum ΔH, Methanococcus jannaschii, Methanothermus fervidus, Pyrobaculum islandicum, Pyrococcus endeavori, Pyrococcus furiosus, Pyrococcus horihoshii, Pyrococcus profundus, Pyrococcus woesei, Pyrodictium occultum, Sulfolobus acidocaldarius, Sulfolobus solfataricus, Thermoanaerobacter thermohydrosulfuricus, Thermococcus celer, Thermococcus fumicolans, Thermococcus gorgonarius, Thermococcus kodakaraensis KOD1, Thermococcus litoralis, Thermococcus peptonophilus, Thermococcus sp. 9° N-7, Thermococcus sp. TY, Thermococcus stetteri, Thermococcus zilligii, Thermoplasma acidophilum, Thermus brokianus, Thermus caldophilus GK24, Thermus flavus, Thermus rubens, or a mutant thereof

8. The thermostable enzyme composition according to claim 1, wherein incubation of said thermostable enzyme composition in an aqueous buffer, formulated to about pH 7 to about pH 8 at 25° C., at a temperature greater that about 50° C. results in at least a two-fold increase in enzyme activity in less than about 20 minutes.

9. The thermostable enzyme composition according to claim 1, wherein the thermostable enzyme has been modified by a carboxylic acid modifier reagent described by the formula: 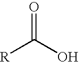
wherein R is a hydrogen, a substituted or unsubstituted phenyl group, a substituted or unsubstituted cycloalkyl group, a substituted or unsubstituted heteroaromatic group, or a substituted or unsubstituted alkyl group.

10. The thermostable enzyme composition according to claim 9, wherein said carboxylic acid modifier reagent is citraconic acid or cis-aconitic acid.

11. A method for reversibly inactivating a thermostable enzyme, comprising
(a) reacting a zero-length cross-linker compound with a carboxylic acid modifier reagent of the formula: 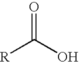
wherein R is hydrogen, a substituted or unsubstituted phenyl group, a substituted or unsubstituted cycloalkyl group, a substituted or unsubstituted heteroaromatic group, or a substituted or unsubstituted alkyl group; and
(b) reacting said activated carboxylic acid modifier reagent with a thermostable enzyme to reversibly inactivate the thermostable enzyme.

12. The method according to claim 11, wherein the zero-length cross-linker provides an ester with the carboxylic acid modifier reagent.

13. The method according to claim 11, wherein the zero-length cross-linker compound is a carbodiimide compound, Woodward's Reagent K, N,N′-Carbonyl Diimidazole, TSTU (O-(N-succinimidyl)-N,N,N′,N′-tetramethyluronium tetrafluoroborate), BTU (O-benzotriazol-1-yl)-N,N,N′,N′-tetramethyluronium hexafluorophosphate), TBTU (2-(1H-benzotriazo-1-yl)-1,1,3,3-tetramethyluronium hexafluorophosphate), TFFH (N,N′, N″, N′″-tetramethyluronium 2-fluoro-hexafluorophosphate), PyBOP (benzotriazole-1-yl-oxy-tris-pyrrolidino-phosphonium hexafluorophosphate), EEDQ (2-ethoxy-1-ethoxycarbonyl-1,2-dihydro-quinoline), DIPCDI (diisopropylcarbodiimide), MSNT (1-(mesitylene-2sulfonyl)-3-nitro-1H-1,2,4-triazole), or a triisopropylbenzenesulfonyl chloride.

14. The method according to claim 13, wherein the carbodiimide compound is 1-ethyl-3-(3-dimethylaminopropyl)carbodiimide hydrochloride (EDC), 1-Cyclohexyl-3-(2-morpholinoethyl)carbodiimide (CMC), dicyclohexylcarbodiimide (DCC), or Diisopropyl carbodiimide (DIC).

15. The method according to claim 11, wherein said carboxylic acid modifier reagent is citraconic acid or cis-aconitic acid.

16. The method according to claim 11, wherein said thermostable enzyme is a thermostable DNA polymerase, a thermostable RNA polymerase, a thermostable RNase H, a thermostable nuclease, or a thermostable DNA ligase, a thermostable reverse transcriptase, a thermostable RecA, a thermostable helicase.

17. The method according to claim 11, wherein said thermostable enzyme is a thermostable polymerase.

18. The method according to claim 11, wherein said thermostable polymerase is a thermostable DNA polymerase.

19. The method according to claim 11, wherein said thermostable polymerase is a thermostable RNA polymerase.

20. The method according to claim 11, wherein said thermostable enzyme is a thermostable nuclease.

21. The method according to claim 11, wherein said thermostable enzyme is derived from Thermus acquaticus, Thermus thermophilus, Thermatoga maritime, Aeropyrum pernix, Aquifex aeolicus, Archaeglobus fulgidus, Bacillus caldotenax, Carboxydothermus hydrogenformans, Methanobacterium thermoautotrophicum ΔH, Methanococcus jannaschii, Methanothermus fervidus, Pyrobaculum islandicum, Pyrococcus endeavori, Pyrococcus furiosus, Pyrococcus horihoshii, Pyrococcus profundus, Pyrococcus woesei, Pyrodictium occultum, Sulfolobus acidocaldarius, Sulfolobus solfataricus, Thermoanaerobacter thermohydrosulfuricus, Thermococcus celer, Thermococcus fumicolans, Thermococcus gorgonarius, Thermococcus kodakaraensis KOD1, Thermococcus litoralis, Thermococcus peptonophilus, Thermococcus sp. 9° N-7, Thermococcus sp. TY, Thermococcus stetteri, Thermococcus zilligii, Thermoplasma acidophilum, Thermus brokianus, Thermus caldophilus GK24, Thermus flavus, Thermus rubens, or a mutant thereof

22. A method for primer extension, comprising
(a) producing a primer extension reaction mixture by combining:
(i) a sample comprising a target nucleic acid:
(ii) a first primer complementary to the target nucleic acid; and
(iii) a thermostable polymerase composition of claim 3; and
(b) incubating said primer extension reaction mixture to a temperature greater than about 50° C. for a period of time sufficient to activate said thermostable DNA polymerase composition so that said polymerase produces primer extension products from said first primer and said target nucleic acid.

23. The method according to claim 22, wherein said primer extension reaction mixture further comprises a second primer complementary to the target nucleic acid.

24. The method according to claim 23, wherein said method is a method of amplifying said target nucleic acid.

25. The method according to claim 22, wherein said thermostable polymerase is a thermostable DNA polymerase.

26. The method according to claim 22, wherein said thermostable polymerase is a thermostable RNA polymerase.

27. The method according to claim 22, wherein said thermostable polymerase is derived from Thermus acquaticus, Thermus thermophilus, Thermatoga maritime, Aeropyrum pernix, Aquifex aeolicus, Archaeglobus fulgidus, Bacillus caldotenax, Carboxydothermus hydrogenformans, Methanobacterium thermoautotrophicum ΔH, Methanococcus jannaschii, Methanothermus fervidus, Pyrobaculum islandicum, Pyrococcus endeavori, Pyrococcus furiosus, Pyrococcus horihoshii, Pyrococcus profundus, Pyrococcus woesei, Pyrodictium occultum, Sulfolobus acidocaldarius, Sulfolobus solfataricus, Thermoanaerobacter thermohydrosulfuricus, Thermococcus celer, Thermococcus fumicolans, Thermococcus gorgonarius, Thermococcus kodakaraensis KOD1, Thermococcus litoralis, Thermococcus peptonophilus, Thermococcus; sp. 9° N-7, Thermococcus sp. TY, Thermococcus stetteri, Thermococcus zilligii, Thermoplasma acidophilum, Thermus brokianus, Thermus caldophilus GK24, Thermus flavus, Thermus rubens, or a mutant thereof.

28. A primer extension reaction mixture, comprising:
(a) a first primer;
(b) nucleotides; and
(c) a thermostable enzyme composition of claim 3.

29. The primer extension reaction mixture according to claim 28, wherein said mixture further comprises a second primer.

30. The primer extension reaction mixture according to claim 28, wherein said nucleotides are ribonucleotides.

31. The primer extension reaction mixture according to claim 28, wherein said nucleotides are deoxyribonucleotides.

32. The primer extension reaction mixture according to claim 28, wherein said thermostable polymerase is a thermostable DNA polymerase.

33. The primer extension reaction mixture according to claim 28, wherein said thermostable polymerase is a thermostable RNA polymerase.

34. The primer extension reaction mixture according to claim 28, wherein said thermostable polymerase is derived from Thermus acquaticus, Thermus thermophilus, Thermatoga maritime, Aeropyrum pernix, Aquifex aeolicus, Archaeglobus fulgidus, Bacillus caldotenax, Carboxydothermus hydrogenformans, Methanobacterium thermoautotrophicum ΔH, Methanococcus jannaschii, Methanothermus fervidus, Pyrobaculum islandicum, Pyrococcus endeavori, Pyrococcus furiosus, Pyrococcus horihoshii, Pyrococcus profundus, Pyrococcus woesei, Pyrodictium occultum, Sulfolobus acidocaldarius, Sulfolobus solfataricus, Thermoanaerobacter thermohydrosulfuricus, Thermococcus celer, Thermococcus fumicolans, Thermococcus gorgonarius, Thermococcus kodakaraensis KOD1, Thermococcus litoralis, Thermococcus peptonophilus, Thermococcus sp. 9° N-7, Thermococcus sp. TY, Thermococcus stetteri, Thermococcus zilligii, Thermoplasma acidophilum, Thermus brokianus, Thermus caldophilus GK24, Thermus flavus, Thermus rubens, or a mutant thereof

35. A kit comprising a thermostable enzyme composition of claim 1.

36. The kit according to claim 35, wherein said thermostable enzyme is a thermostable DNA polymerase, a thermostable RNA polymerase, a thermostable RNase H, a thermostable nuclease, or a thermostable DNA ligase, a thermostable reverse transcriptase, a thermostable RecA, a thermostable helicase.

37. The kit according to claim 35, wherein said thermostable enzyme is a thermostable polymerase.

38. The kit according to claim 35, wherein said thermostable polymerase is a thermostable DNA polymerase.

39. The kit according to claim 35, wherein said thermostable polymerase is a thermostable RNA polymerase.

40. The kit according to claim 35, wherein said thermostable enzyme is a thermostable nuclease.

41. The kit according to claim 35, wherein said thermostable DNA polymerase is derived from Thermus acquaticus, Thermus thermophilus, Thermatoga maritime, Aeropyrum pernix, Aquifex aeolicus, Archaeglobus fulgidus, Bacillus caldotenax, Carboxydothermus hydrogenformans, Methanobacterium thermoautotrophicum ΔH, Methanococcus jannaschii, Methanothermus fervidus, Pyrobaculum islandicum, Pyrococcus endeavori, Pyrococcus furiosus, Pyrococcus horihoshii, Pyrococcus profundus, Pyrococcus woesei, Pyrodictium occultum, Sulfolobus acidocaldarius, Sulfolobus solfataricus, Thermoanaerobacter thermohydrosulfuricus, Thermococcus celer, Thermococcus fumicolans, Thermococcus gorgonarius, Thermococcus kodakaraensis KOD1, Thermococcus litoralis, Thermococcus peptonophilus, Thermococcus sp.9N-7, Thermococcus sp. TY, Thermococcus stetteri, Thermococcus zilligii, Thermoplasma acidophilum, Thermus brokianus, Thermus caldophilus GK24, Thermus flavus, Thermus rubens, or a mutant thereof.


SEQUENCE LISTING (35 sequences)

SEQ ID NO: 1 - DNA, 27 bp, Artificial Sequence
  Primer
ggaatgagtccactttaaatcctttaa

SEQ ID NO: 2 - DNA, 22 bp, Artificial Sequence
  Primer
cgaggatccattggagggcaag

SEQ ID NO: 3 - DNA, 64 bp, Artificial Sequence
  Primer
cttgccctccaatggatcctcgttaaaggatttaaagtggactcattccaattacagggcctcg

SEQ ID NO: 4 - DNA, 19 bp, Artificial Sequence
  Primer
cgaggccctgtaattggaa

SEQ ID NO: 5 - DNA, 17 bp, Artificial Sequence
  Primer
cggctgctggcaccaga

SEQ ID NO: 6 - DNA, 24 bp, Artificial Sequence
  Primer
tgctgaatttccatctctgagttc

SEQ ID NO: 7 - DNA, 21 bp, Artificial Sequence
  Primer
gcaggattcagtgccagaaag

SEQ ID NO: 8 - DNA, 14 bp, Artificial Sequence
  Primer
taccacgctttttc

SEQ ID NO: 9 - DNA, 27 bp, Artificial Sequence
  Primer
ggcaaagaacagaagtaaaatccagaa

SEQ ID NO: 10 - DNA, 24 bp, Artificial Sequence
  Primer
cagtttcacagtgaaagttggcaa

SEQ ID NO: 11 - DNA, 13 bp, Artificial Sequence
  Primer
tgcctcaagcagc

SEQ ID NO: 12 - DNA, 19 bp, Artificial Sequence
  Primer
tgggcctgaccactccttt

SEQ ID NO: 13 - DNA, 18 bp, Artificial Sequence
  Primer
tgcgatcccgcttgtgat

SEQ ID NO: 14 - DNA, 13 bp, Artificial Sequence
  Primer
tgcccagccccag

SEQ ID NO: 15 - DNA, 19 bp, Artificial Sequence
  Primer
caggtggagaccctgagaa

SEQ ID NO: 16 - DNA, 21 bp, Artificial Sequence
  Primer
acacctttggtcactccaaat

SEQ ID NO: 17 - DNA, 20 bp, Artificial Sequence
  Primer
tcccagagctcccagggtcc

SEQ ID NO: 18 - DNA, 19 bp, Artificial Sequence
  Primer
gcggagggaagctcatcag

SEQ ID NO: 19 - DNA, 22 bp, Artificial Sequence
  Primer
ccctagtctcagaccttcccaa

SEQ ID NO: 20 - DNA, 24 bp, Artificial Sequence
  Primer
ccacgagctgagtgcgtcctgtca

SEQ ID NO: 21 - DNA, 22 bp, Artificial Sequence
  Primer
cattcctctgcagcacttcact

SEQ ID NO: 22 - DNA, 22 bp, Artificial Sequence
  Primer
cggttcagtccacataatgcat

SEQ ID NO: 23 - DNA, 15 bp, Artificial Sequence
  Primer
caaatgagcattagc

SEQ ID NO: 24 - DNA, 26 bp, Artificial Sequence
  Primer
gaaacgcatctcactgtcattctatt

SEQ ID NO: 25 - DNA, 23 bp, Artificial Sequence
  Primer
caccatacttcatggcaaggact

SEQ ID NO: 26 - DNA, 15 bp, Artificial Sequence
  Primer
caccattagatcctg

SEQ ID NO: 27 - DNA, 15 bp, Artificial Sequence
  Primer
caccattaggtcctg

SEQ ID NO: 28 - DNA, 21 bp, Artificial Sequence
  Primer
gaggtttcactggcttgtgct

SEQ ID NO: 29 - DNA, 33 bp, Artificial Sequence
  Primer
catgagacatttatctaatgattttttcttatt

SEQ ID NO: 30 - DNA, 13 bp, Artificial Sequence
  Primer
ccatgcgttagcc

SEQ ID NO: 31 - DNA, 15 bp, Artificial Sequence
  Primer
ccatgggttagccaa

SEQ ID NO: 32 - DNA, 24 bp, Artificial Sequence
  Primer
tgctgaatttccatctctgagttc

SEQ ID NO: 33 - DNA, 21 bp, Artificial Sequence
  Primer
gcaggattcagtgccagaaag

SEQ ID NO: 34 - DNA, 14 bp, Artificial Sequence
  Primer
taccacgctttttc

SEQ ID NO: 35 - DNA, 16 bp, Artificial Sequence
  Primer
tgtaccactctttttc